(19) United States
(12) Patent Application Publication
(10) Pub. No.: US 2026/0041460 A1
(43) Pub. Date: Feb. 12, 2026

(54) METHODS AND ARRANGEMENTS FOR DYNAMIZING BONE ALIGNMENT DEVICES

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Andrew P. Noblett, Bartlett, TN (US); Paul Bell, Vestavia Hills, AL (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/998,328

(22) PCT Filed: Sep. 13, 2023
(86) PCT No.: PCT/US23/32606

§ 371 Date: Jan. 24, 2025

Related U.S. Application Data
(60) Provisional application No. 63/406,504, filed on Sep. 14, 2022.

(51) Int. Cl. A61B 17/66; A61B 17/62; A61B 34/00; A61B 34/10; A61B 90/00

(52) U.S. Cl. CPC A61B 17/66; A61B 17/62; A61B 34/10; A61B 34/25; A61B 90/06; A61B 2034/254; A61B 2090/064

(57) ABSTRACT

Logic may interact with a user to determine a pattern of micromotions to associate with an adjustment schedule. Logic may interact with the user via a user interface element to determine a rate of micromotions to associate with the adjustment schedule. Logic may associate the set of instructions with the adjustment schedule. Logic may cause the transmission of the set of instructions to a patient device for execution during treatment in conjunction with the adjustment schedule. And logic may cause transmission of communications to one or more motor controller circuits of the bone alignment device to perform the micromotions based on execution of the instructions to apply micromotions to the portion of the adjustment schedule via an automated bone alignment device.

### CROSS-REFERENCE TO RELATED APPLICATIONS

This is a non-provisional of, and claims the benefit of the filing date of, U.S. provisional patent application number 63/406,504, filed Sep. 14, 2022, entitled “Methods and Arrangements for Dynamizing Bone Alignment Devices,” the entirety of which application is incorporated by reference herein.

### FIELD OF THE DISCLOSURE

The present disclosure relates generally to orthopedic devices, systems, and methods to dynamize a bone alignment device to improve bone regeneration.

### BACKGROUND OF THE DISCLOSURE

Bone alignment devices, also referred to as bone fixators, are very effective clinically for correction of orthopedic deformities because of the concept of distraction osteogenesis and the loading that is possible during treatment. It is well documented that bone responds to mechanical loads, particularly periods of intermittent loading. Studies have shown that bone reacts differently to different rates, magnitudes, and patterns of loading.

Software for bone alignment devices may determine a correction of a bone broke or cut into bone segments, and by extension, a correction path and a correction rate for healing the bone segments. A surgeon enters parameters in the software to define the patient's orthopedic deformity, the hardware applied to the patient, the hardware location relative to the deformity, and the desired duration/distraction rate of the correction. Based on the surgeon's inputs, the software determines the final, corrected state of the patient's bone alignment device and provides an adjustment schedule (also known as a prescription) which allows the bone alignment device to be controllably manipulated from the initial postoperative state to the final corrected state.

Orthopedic deformities are three dimensional problems and are typically described quantitatively with six deformity parameters. The deformity parameters are usually described as anteroposterior (AP) view translation, AP view angulation, sagittal (LAT) view translation, LAT view angulation, axial view translation, and axial view angulation. Deformity parameters may be evaluated from medical images, such as AP and Lateral radiographs or three-dimensional (3D) imaging modalities, and clinical evaluations.

Software applications for bone alignment devices can calculate the position of the hardware and bone segments to a high degree of accuracy throughout the adjustments defined by the prescription. Surgeons can use software applications with bone alignment devices pre-operatively, intra-operatively, and/or post-operatively. Most modern bone alignment device software applications are web-based.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Some examples include methods and arrangements to dynamize a bone alignment device. The methods and arrangements may include providing an adjustment schedule to align a static bone segment and a moving bone segment. The adjustment schedule may include a series of waypoints for a point on the moving bone segment. The methods and arrangements may include interacting with a user via a first user interface element to determine a pattern of micromotions to associate with the adjustment schedule and interacting with the user via a second user interface element to determine a rate of micromotions to associate with the adjustment schedule. The methods and arrangements may include determining a set of instructions for micromotions based on input from a user and boundaries of a correction path associated with the adjustment schedule. The set of instructions for the micromotions may establish the rate for the micromotions and the pattern for the micromotions for the dynamizing the adjustment schedule.

The methods and arrangements may include associating the set of instructions with the adjustment schedule; and causing the transmission of the set of instructions to a device for execution during treatment according to the adjustment schedule. The execution of the set of instructions may cause the bone alignment device to automatically move the point of the moving bone segment in micromotions within the boundaries of the correction path in accordance with the set of instructions.

Any preceding or subsequent example may further include interacting with the user via a third user interface element to determine a rest interval between application of the micromotions. In any preceding or subsequent example, the rest interval between application of the micromotions includes a schedule. In any preceding or subsequent example, the schedule may include a time of day.

In any preceding or subsequent example, the rest interval between application of the micromotions includes a period of time. In any preceding or subsequent example, periods of micromotions and rest intervals occur with varying durations throughout a period of time.

Any preceding or subsequent example may further include receiving sensor data and adjusting micromotions in response. Adjusting the micromotions may include adjusting the rate of the micromotions, a magnitude of the micromotions, a direction of the micromotions, or a combination thereof. Any preceding or subsequent example may further include receiving sensor data and stopping micromotions in response to the sensor data, wherein the sensor data reaches or exceeds a threshold for the sensor data. In any preceding or subsequent example, the sensor data may include a force, a strain, a power usage, or a combination thereof.

Any preceding or subsequent example may further include interacting with the user via a fourth user interface element to determine a portion of the adjustment schedule within which to integrate the set of instructions for the micromotions. In any preceding or subsequent example, the portion of the adjustment schedule includes a schedule. In any preceding or subsequent example, the portion of the adjustment schedule includes a pre-treatment portion, an initial treatment portion, a middle treatment portion, an end treatment portion, a post-treatment portion, or a combination thereof.

In any preceding or subsequent example, the pattern of micromotions may include a forward direction and a reverse direction. The forward direction may be a direction along a mechanical axis of the static bone segment from a starting point. The starting point may be a waypoint in the adjustment schedule. The reverse direction may be a direction opposite of the forward direction to move the point on a moving bone segment along the mechanical axis of the static bone segment back to the starting point.

In any preceding or subsequent example, the pattern of micromotions may include a first direction and a second direction. The first direction may be in any direction and, in any preceding or subsequent example, may not be parallel to the mechanical axis of the static bone segment or the moving bone segment. The first direction is away from a starting point and the second direction is a direction opposite of the first direction to move the point on the moving bone segment back to the starting point.

In any preceding or subsequent example, the pattern of micromotions includes a first direction and a second direction, wherein the first direction is away from a starting point and the second direction is a direction toward a waypoint of the adjustment schedule that is subsequent to the starting point. In other words, in such examples, the micromotions may be incorporated into the correction path between two waypoints. In any preceding or subsequent example, the pattern of micromotions may include a first direction and a second direction, wherein the first direction is not parallel to a mechanical axis of the static bone segment.

In any preceding or subsequent example, the pattern of micromotions may include an orbital pattern. The orbital pattern may return the point on the moving bone segment to the starting point. In any preceding or subsequent example, the orbital pattern moves the point on the moving bone segment to a subsequent waypoint. In any preceding or subsequent example, the orbital pattern moves the point on the moving bone segment to an intermediate point that is not the starting point and not the subsequent waypoint. In such examples, a subsequent set of micromotions may return the point on the moving bone segment to the starting point or move the point on the moving bone segment to the subsequent waypoint.

In any preceding or subsequent example, the pattern of micromotions moves the point on the moving bone segment to a subsequent waypoint. In any preceding or subsequent example, the pattern of micromotions unevenly apply force and strain to a side of the static bone segment and a side of the moving bone segment to stimulate bone growth on one side of the patient anatomy. In any preceding or subsequent example, pattern of micromotions apply a torsional micromotion to the static bone segment and/or the moving bone segment.

Any preceding or subsequent example may further include applying micromotions to the portion of the adjustment schedule via an automated bone alignment device in response to execution of the set of instructions. The portion may include a pre-treatment portion, an initial treatment portion, a middle treatment portion, an end treatment portion, a post-treatment portion, or a combination thereof.

Any preceding or subsequent example include methods and arrangements to dynamize a bone alignment device. The methods and arrangements may include receiving an adjustment schedule with a set of instructions for micromotions for a treatment plan for a bone alignment device. The methods and arrangements may include executing the set of instructions prior to performing adjustments of the adjustment schedule, during one or more portions of the entire treatment plan, after performing the adjustment schedule, or combination thereof.

Note that, as used herein, the prescription is an adjustment schedule and the treatment plan includes the prescription as well as instructions for micromotions to perform in conjunction with the prescription. The instructions for the micromotions may include one or more sets of micromotions and each set of micromotions may be associated with a rate, a pattern, and a rest interval. The rest interval may include a period of time to pause micromotions and/or a schedule during which to implement micromotions. For instance, the treatment plan may include an adjustment schedule along with instructions to implement a set of micromotions having, e.g., an orbital pattern at a rate of 1 Hz between 6 post meridiem (pm) and 8 pm each day with a 30-minute rest period between sets of micromotions. The magnitude of the orbital micromotions may be calculated based on the boundaries of the correction path.

In any preceding or subsequent example, the treatment plan may include an adjustment schedule along with instructions to implement a set of micromotions having forward and reverse direction pattern along a mechanical axis of the static bone segment at a rate of 0.75 Hz with a magnitude of 0.1 millimeter (mm) between 2 pm and 3 pm for the first 11 days of the adjustment schedule with a 20-minute rest period between each set of micromotions. In any preceding or subsequent example, the correction logic circuitry may determine the correction path and the prescription taking into consideration one or more sets of micromotions at user specified rest intervals. In such examples, the correction logic circuitry may maximize the magnitude of the micromotions based on a default setting for the magnitude and/or a preference setting by the user.

In any preceding or subsequent example, the correction logic circuitry may cause transmission of communications with one or more motors of the bone alignment device to perform the micromotions based on execution of the instructions. In any preceding or subsequent example, the correction logic circuitry may display a confirmation of receipt of instructions by the bone alignment device, or one or more components (such as automatic struts) thereof. In any preceding or subsequent example, the correction logic circuitry may display sensor data associated with the operation of the bone alignment device. In any preceding or subsequent example, the correction logic circuitry may display a status of performance of micromotions. And, in any preceding or subsequent example, the correction logic circuitry may cause transmission of alerts related to performance of micromotions to a device such as a patient device or a server.

Examples of the present disclosure provide numerous advantages. For example, the correction logic circuitry may advantageously perform operations such as providing a graphical user interface to add micromotions to adjustment schedule of a prescription to (dynamize) a bone alignment device and interacting with a user via a first user interface element to determine a pattern of micromotions to associate with the adjustment schedule. The correction logic circuitry may advantageously perform operations such as interacting with the user via a second user interface element to determine a rate of micromotions to associate with the adjustment schedule. The correction logic circuitry may advantageously perform operations such as determining a set of instructions for micromotions based on input from a user and boundaries of a correction path associated with the adjustment schedule, wherein the set of instructions for the micromotions establish the rate for the micromotions and the pattern for the micromotions. The correction logic circuitry may advantageously perform operations such as associating the set of instructions with the adjustment schedule and causing the transmission of the set of instructions to a device for execution during treatment according to the adjustment schedule. The execution of the set of instructions may advantageously cause the bone alignment device to automatically move the point of the moving bone segment in micromotions within the boundaries of the correction path in accordance with the set of instructions. And the correction logic circuitry may advantageously perform operations such as applying micromotions to the portion of the adjustment schedule via an automated bone alignment device in response to execution of the set of instructions. The portion may include a pre-treatment portion, an initial treatment portion, a middle treatment portion, an end treatment portion, a post-treatment portion, or a combination thereof. Applying micromotions may involve, for instance, sending commands to one or more motor controller circuits of the bone alignment device to perform micromotions before, during, or after adjusting the bone alignment device in accordance with an adjustment on the adjustment schedule.

Study results show improvements to bone regenerate when dynamizing the bone alignment device. Each of the approaches to dynamization described herein may have their own merit in specific circumstances. Some examples may, for instance, advantageously implement dynamization after corrections are completed (completion of the adjustments of the prescription) during the consolidation phase of treatment of the patient. Other examples may, for instance, advantageously perform a reverse dynamization that applies micromotions during an early portion of the adjustment schedule and does not apply micromotions during a final portion of the adjustment schedule.

With an automated bone alignment device, any of these approaches to dynamization may advantageously be pursued based on the preference of the user (e.g., surgeon) in accordance with the user's assessment of the most advantageous portions of the adjustment schedule during which to apply the micromotions. Via the correction logic circuitry of a device such as a patient device or a device assigned to a patient, dynamization may be applied for a portion of the treatment plan (beginning, middle, or end) or throughout the entire treatment plan based on the preference of the user and in accordance with the instructions generated for the micromotions.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1A illustrates an example of a system to dynamize a bone alignment device for treating a patient;

FIGS. 1B-F illustrate examples of anteroposterior (AP) view and lateral (LAT) view outline images of a tibia aligned and misaligned;

FIGS. 2A-H illustrate examples of a user interface to provide input data to correction logic circuitry;

FIG. 2I illustrates an example of a prescription with daily adjustments;

FIG. 3A illustrates an example of a graphical user interface of correction logic circuitry such as the correction logic circuitry discussed in conjunction with FIG. 1A to determine micromotions to associate with a prescription such as the prescription shown in FIG. 2I;

FIG. 3B illustrates an example of a micromotion schedule associated with a prescription such as the prescription shown in FIG. 2I;

FIG. 3C illustrates an example of a patient device such as the patient device illustrated in FIG. 1A;

FIG. 3D illustrates an example of a bone alignment device with geared motor assemblies for automatic adjustments according to a prescription such as the prescription shown in FIG. 2I;

FIG. 3E illustrates an example of an automatic strut comprising a strut coupled with a geared-motor assembly for automatic adjustments according to a prescription such as the prescription shown in FIG. 2I;

FIG. 3F illustrates an example of a motor controller circuit for a geared motor assembly coupled with a bone alignment device for automatic adjustments according to a prescription such as the prescription shown in FIG. 2I;

FIGS. 4A-G illustrate examples of patterns of micromotions associated with a prescription such as the prescription shown in FIG. 2I;

FIGS. 5A-B illustrate examples of flowcharts to generate instructions to implement micromotions and to implement micromotions in conjunction with a prescription such as the prescription shown in FIG. 2I;

FIG. 6 depicts an example of a system including a multiple-processor platform, a chipset, buses, and accessories the server, HCP device, and the patient device shown in FIG. 1A; and

FIGS. 7-8 depict examples of a storage medium and a computing platform such as the server, HCP device, and the patient device shown in FIG. 1A and FIG. 6.

The drawings are not necessarily to scale. The drawings are merely representations, not intended to portray specific parameters of the disclosure. The drawings are intended to depict various examples of the disclosure, and therefore are not to be considered as limiting in scope. In the drawings, like numbering represents like elements.

### DETAILED DESCRIPTION

Surgeons may use software to determine any hardware adjustments necessary for bone alignment devices to achieve a correction and, by extension, the correction path and correction rate of the bone alignment device during treatment. A surgeon enters parameters in the software to define the patient's deformity, the hardware applied to the patient, the hardware location(s) relative to the deformity, and the desired duration/distraction rate of the correction. Based on the surgeon's inputs, the bone alignment device software determines the final, corrected state of the patient's bone alignment device and provides an adjustment schedule, which will allow the bone alignment device to be controllably manipulated from the initial postoperative state to the final corrected state. The bone is divided into bone segments. As discussed herein, each pair of bone segments include a first bone segment designated as a fixed or static bone segment and a second bone segment designated as a moving bone segment. Prescriptions to correct deformities, as discussed herein, focus on a correction path for correcting two bone segments. If multiple bone segments are being treated, examples may correct each pair of bone segments via a separate prescription.

During correction, the moving bone segment may move according to hardware adjustments, e.g., adjustments to the lengths of one or more struts of a bone alignment device. The path that the moving bone segment follows during the correction is referred to as the correction path. The schedule of hardware adjustments is referred to as a prescription or an adjustment schedule.

Note that, in some situations, the adjustments are effectively movements of the bone segments with respect to each other but are referred to herein as movement of the moving bone segment for the purposes of describing the generation and implementation a treatment plan with micromotions. In other words, discussions herein describe movement of the moving bone segment and consider the static bone segment to be fixed for clarity, but the treatment plans discussed herein apply equally to situations in which the treatment plan may cause movement of the static bone segment, the moving bone segment, or both.

Bone alignment device software may dictate correction paths by prioritizing an even distribution of hardware adjustments during a correction. Such bone alignment device software calculates distances between the initial and final positions of a point on the moving bone segment and the initial and final states of the hardware. Bone alignment device software may, alternatively, calculate the distance that a point on a specified anatomic structure of interest travels during the correction. No matter which type of point is used, the calculated travel distance of the point is divided by the maximum allowable correction rate in units of millimeters per day (mm/day) to determine the number of days for the correction. Angular correction rate parameters control the degrees of rotation per day of correction (deg/day) and may also be considered in the calculation of the number of days required for the correction. These maximum allowable correction rates may be referred to as the correction path boundaries.

Bone alignment device software may distribute the hardware adjustment required to correct the deformity according to the calculated number of days required for the correction. Bone alignment device software may check the calculated movements of the bone segments against rate limiting inputs for all adjustments (maximum allowed translation per day and/or maximum allowable degrees of rotation per day). If any of the calculated adjustments exceed defined rate limits, then the bone alignment device software may add additional days to the adjustment schedule to ensure that limits are always maintained.

Some examples may allow dynamization, controlled micromotions, to be incorporated into to a treatment plan and similarly compare micromotions for dynamizing the bone alignment device against rate limiting inputs (maximum allowed translation per day and/or maximum allowable degrees of rotation per day) to ensure that the micromotions do not exceed the boundaries of the correction path. In some examples, the magnitude and/or pattern of the micromotions are limited to avoid exceeding the boundaries of the correction path. For instance, in some examples, the user selects the pattern of the micromotions and the correction logic circuitry calculates a magnitude for the micromotions to limit movements of the moving bone segment to remain within the boundaries of the correction path. In some examples, the user may select the pattern and the magnitude, and the user may also select a preference to indicate that correction logic circuitry should limit the magnitude for the micromotions to limit movements of the moving bone segment to remain within the boundaries of the correction path. Alternatively, the user may select a preference to indicate that that the correction logic circuitry should adjust the pattern for the micromotions to limit movements of the moving bone segment to remain within the boundaries of the correction path. In further examples, the correction logic circuitry may present alternatives such as a reduced magnitude for the pattern or a modified pattern for the micromotions to maintain the movements of the moving bone segment within the boundaries of the correction path.

Other bone alignment device software may dictate the correction path of a bone alignment device according to the path of the anatomy. The bone alignment device software may calculate a line between the initial and final locations of a point on the moving bone segment (or a point on a specified anatomic structure as described above). The bone alignment device software may then calculate hardware adjustments so that the moving bone segment follows the linear path within a tolerance band (or correction path boundaries) according to the specified rate of correction. If any of the calculated adjustments exceed defined rate limits, then additional days may be added to the adjustment schedule to ensure that limits are always maintained.

Some bone alignment device software may allow users to adjust the correction path. For instance, the bone alignment device software may offer the user an option to divide an adjustment schedule into two phases. The first phase may correct a specified axial translation deformity (lengthening or shortening) and a second phase may correct the remaining deformity. Dividing an adjustment schedule into two phases allows the user to distract the bone segments for clearance before correcting translation and angulation in other directions.

Some bone alignment device software may offer customization of the correction path through waypoints of correction. Waypoints of a correction path may be defined as any specified orientation or position of the moving bone fragment that may be achieved during the correction. The waypoints may allow users to break a correction into multiple phases and correct specified amounts of angulation and translation within each phase. The bone alignment device software may include inputs via a graphical user interface or other user interface, to allow the user to specify the numerical value and direction of the angulation or translation that is to be corrected in each phase.

Automated bone alignment devices open new opportunities for bone alignment. Automated or motorized bone alignment devices accomplish the prescribed hardware adjustments by any combination of motors and electronics. One major advantage of automated bone alignment devices is that patient compliance, which is crucial to traditional bone alignment devices requiring adjustment, is no longer a concern. Automated bone alignment devices self-adjust according to their programmed schedule of adjustments. A second major advantage of automated bone alignment devices is that the devices can achieve a granularity for adjustments that is not possible with traditional bone alignment devices.

In some examples, during the correction phase of treatment with a bone alignment device, the bone alignment device may manipulate one bone segment in relation to another bone segment. Correction logic circuitry may guide the manipulation of the bone segments to generate a prescription of adjustments to achieve a proper correction based on hardware, deformity, and rate defining inputs. The correction logic circuitry may also incorporate micromotions, dynamization, as a pretreatment, during correction, and/or after correction of the deformity with the bone alignment device. In some examples, the correction logic circuitry may implement micromotions throughout an entire treatment plan.

The correction logic circuitry may interact with a user graphically or via keystrokes to graphically define a correction path on 2D images or a 3D image for correction and/or interact with a user to define waypoints or stages of the correction path for deformity correction. The waypoints may define the correction path in stages of correction from an initial postoperative state to a final corrected state. The final corrected state may optionally leave some remaining deformity of the bone segments.

The prescription may include a list of the waypoints of the correction path in the form of adjustments to be made to a bone alignment device as well as the timing for the adjustments. For instance, a prescription may be an adjustment schedule comprising a listing of days, such as 28 days, which represents the duration of the treatment plan and adjustments or settings for the bone alignment device for, e.g., each day of the prescription. Each adjustment may include an adjustment or setting for, e.g., one or more struts of the bone alignment device.

The correction logic circuitry may interact with a user such as a surgeon to determine parameters of micromotions to incorporate in the treatment plan in conjunction with the adjustment schedule to dynamize the bone alignment device. The correction logic circuitry may interact with the user before, during, and/or after the creation of the adjustment schedule of the treatment plan to define micromotions to perform during execution of the treatment plan. For instance, prior to the generation of an adjustment schedule, the correction logic circuitry may interact with the user to generally define when micromotions are implemented with respect to the adjustment schedule to dynamize the bone alignment device. In such examples, the correction logic circuitry may generate instructions for the micromotions during the creation of the adjustment schedule. In other examples, the user may interact with the correction logic circuitry to implement the micromotions after completion of the adjustment schedule.

The correction logic circuitry may, advantageously, generate a graphical user interface (GUI) with two or more user interface elements to define the parameters for the micromotions. For instance, the graphical user interface may include a user interface element to determine a rate for the micromotions. The rate of micromotions may impact the bone quality after correction of the bone. The user interface element may include one or more settings such as a default setting such as 1 hertz (Hz) and one or more alternative settings such as settings in 0.1 Hz increments between 0.5 Hz and 1.5 Hz or 0.2 Hz and 2 Hz. Note that 1 Hz is defined as 1 cycle per second.

The correction logic circuitry may, advantageously, generate a GUI with a user interface element including one or more settings for a rest interval between the micromotions. For instance, the correction logic circuitry may include a rest interval such as a period of time (e.g., 30 minutes) and/or may include a schedule such as between 6:30 pm and 8 pm. In some examples, the user may interact with one or more user interface elements to schedule micromotions independently for each day of the treatment plan or each week of the treatment plan. For instance, the user may create a calendar and select a schedule for weekdays and a schedule for weekends. In some examples, the user may schedule micromotions between follow-up visits by a doctor to evaluate the progress and/or the patient's experience with the prescription and micromotion schedule.

The correction logic circuitry may also, advantageously, generate a GUI with a user interface element including one or more settings for a pattern for the micromotions. The user may define the micromotions as movements of a moving bone segment with respect to the correction path at the current waypoint, a point on a moving bone segment at the current waypoint, or a point on a static bone segment at the current waypoint. For instance, the user may define micromotions as movements of a point on the moving bone segment to move in a first direction away from the current waypoint and to move the moving bone segment in an opposite direction back to the current waypoint. In some examples, the entire movement in the first direction and the opposite direction are considered one cycle so the forward and reverse movements may occur in one second when the rate is set to 1 Hz.

In some examples, the user may interact with a user interface element of the GUI to define a magnitude of the micromotions such as 0.1 millimeter (mm), 0.2 mm, 0.3 mm, and/or the like for micromotions. In several examples, the correction logic circuitry may implement the micromotions at the magnitudes indicated by the user. In some examples, the correction logic circuitry may limit the magnitude of the micromotions to magnitudes within defined boundaries or limits associated with the correction path. For instance, the boundaries of the correction path may include the maximum translation rate and/or maximum rotation per adjustment, such as per day. In such a case, the micromotions will be allowed as planned in the GUI until continuing the micromotions would cause translations and/or angulations beyond the set limits. Boundaries may also definite the translation/angulation movement limits of micromotions. For example, micromotions may be programmed to never exceed 0.5 mm beyond the prescribed path of the moving bone segment. In some examples, the micromotions may exceed the boundaries by a percentage such, e.g., 1 percent or by, e.g., a translation such as 0.01 mm or a rotation such as 0.01 degrees. In other examples, the correction logic circuitry may adjust the number of days in the prescription schedule to facilitate a specified or selected magnitude of translation and/or rotation via the micromotions to avoid exceeding the set daily boundaries of the prescription.

The correction logic circuitry may define micromotions based on adjustments or settings for, e.g., struts of the bone alignment device. In many examples, the correction logic circuitry may calculate changes to the adjustments or settings for the struts based on the current waypoint and the micromotions defined by the user. In some examples, the correction logic circuitry may also calculate movements to remain within the boundaries of the correction path for the prescription. For instance, the user may set a maximum translation rate and/or a maximum angular rate for movements of the moving bone segment for generation of the prescription. In some examples, the maximum translation rate and/or a maximum angular rate for movements of the moving bone segment may be included with the prescription. In other examples, the correction logic circuitry may receive the maximum translation rate and/or a maximum angular rate for movements through interaction with the user or from another software module employed to create the prescription for the patient.

In some examples, the micromotions may begin at a waypoint of the adjustment schedule as a starting point and return to the next waypoint on the adjustment schedule as the endpoint of the micromotions. In some examples, the micromotions may begin at a first waypoint and end at an intermediate waypoint (not on the adjustment schedule) after a first set of micromotions and then move to the next waypoint on the adjustment schedule after one or more additional sets of micromotions. In some examples, the micromotions may begin at a first waypoint and end at an intermediate waypoint after a first set of micromotions and then return to the first waypoint after one or more additional sets of micromotions.

In some examples, the micromotions may include a pattern of movement that begins at a current waypoint of the adjustment schedule as a starting point and returns to the next waypoint on the adjustment schedule as the endpoint of the micromotions. In some examples, the pattern of micromotions includes a first direction and a second direction, wherein the first direction is not parallel to a mechanical axis of the static bone segment. In some examples, the pattern may include an orbital pattern, a circular pattern, a hexagonal pattern, and/or the like. In some examples, the pattern of micromotions unevenly apply force and strain to a side of the regenerate bone to stimulate bone growth on one side of the patient anatomy. In some examples, the pattern of micromotions apply a torsional micromotion to the regenerate bone.

In some examples, the correction logic circuitry may couple with sensors attached with and/or integrated with one or more components of the bone alignment device. In some examples, the sensor data may trigger an event such as an event to temporarily stop automated micromotions and/or adjustments via the bone alignment device. For instance, the correction logic circuitry may receive sensor data and adjust micromotions in response, wherein adjusting the micromotions includes adjusting the rate of the micromotions, a magnitude of the micromotions, a direction or pattern of the micromotions, or a combination thereof.

After interacting with the user to determine a set of micromotions to apply to the adjustment schedule, the correction logic circuitry may generate instructions representative of the micromotions for execution by a patient device such as a desktop computer, laptop computer, tablet, smart phone, and/or the like. The patient device may then communicate commands for micromotions to the automated bone alignment device as appropriate to implement the prescription along with the micromotions. In some examples the patient device may be replaced by a device operated by a health care professional or other individual.

In some examples, correction logic circuitry may reside on a patient device (such as a device assigned to or owned by the patient) to implement a prescription along with micromotions to dynamize a bone alignment device. The correction logic circuitry may include firmware, an app, or a software application to execute on a processor or processing circuitry of the patient device as well as memory such as registers buffers, processor pipelines, cache, and/or the like within and/or coupled with the processor while executing a portion code of the firmware, app, or software application.

The correction logic circuitry may receive a prescription with a set of instructions for micromotions for a treatment plan for a bone alignment device. In some examples the instructions for micromotions for a treatment plan for a bone alignment device may include indications of predefined micromotions known to the correction logic circuitry as well as indications for the timing or schedule for the micromotions and rest intervals. In other examples, the instructions for micromotions for a treatment plan for a bone alignment device may define movements or adjustments for the bone alignment device by defining the rate, rest interval, pattern, magnitude, and/or the like for the micromotions.

The correction logic circuitry may cause the patient device to transmit communications such as commands to one or more motor controllers of the bone alignment device to perform the micromotions based on execution of the instructions for the micromotions. In some examples, the correction logic circuitry may monitor execution of the commands for the micromotions based on feedback from one or more motor controller circuits and/or one or more sensors coupled with the bone alignment device to verify that micromotions are performed. For examples in which the bone alignment device includes multiple components to adjust for each micromotion, the correction logic circuitry may monitor feedback from the motor controller circuit(s) and/or sensor(s) to verify that physical adjustment of each of the multiple components does occur, avoiding complications related to a failure of one or more of the multiple components. For example, a bone alignment device may include six automatic struts and each of the automatic struts may include a motor controller circuit coupled with a motor, a sensor to monitor some aspect of power consumption, and a sensor to monitor some physical aspect of strut adjustment. The correction logic circuitry of the patient device may wirelessly transmit a command to one or more of the six motor controller circuits to make respective adjustments to the struts in accordance with a set of micromotions. The one or more motor controller circuits may wirelessly respond with feedback confirming execution of the respective commands as well as sensor data collected from the sensors related to power consumption and the physical aspect of strut adjustment. In response to the feedback, the correction logic circuitry may verify that the sensor data reflect execution of the strut adjustments confirmed by the motor controller circuits. If the feedback indicates that one or more adjustments did not occur or may not have occurred, such as feedback indicating zero power consumption by a strut that should have been adjusted, the correction logic circuitry may terminate execution of the micromotions. The correction logic may also terminate execution of the adjustment schedule and may transmit a communication to the patient and/or another person such as a surgeon to indicate an issue with the bone alignment device. For instance, the correction logic circuitry may transmit a message to the patient indicating that the power source of the strut has failed.

In some examples, the correction logic circuitry may display a confirmation of receipt of instructions by the bone alignment device; display sensor data; display a status of performance of micromotions; and cause transmission of alerts related to performance of micromotions. In some examples, the correction logic circuitry may ask the patient to confirm execution of the micromotions prior to instructing the motor controllers to perform the micromotions. In other examples, correction logic circuitry may automatically transmit the instructions to the motor controllers of the bone alignment device to execute of the micromotions in accordance with the schedule and/or rest intervals provided by the user.

While many examples herein discuss and illustrate an exterior bone alignment device for tibia and fibula fractures, examples are applicable to deformations, soft tissue contractures, or fractures of any orthopedic correction area. Furthermore, examples described herein focus primarily on a single fracture that separates a bone into two bone segments, but examples are not limited to a single fracture or osteotomy of, e.g., a tibia or fibula. Examples may address each pair of bone segments separately and the bone segments may be part of any bone. For instance, a tibia may be fractured into three bone segments, i.e., a first bone segment, a second bone segment, and a third bone segment. Such examples may identify the deformity of the first bone segment and the second bone segment and identify the deformity of the third bone segment with respect to the second bone segment.

Logic circuitry herein refers to a combination of hardware and code to perform functionality. For instance, the logic circuitry may include circuits such as processing circuits to execute instructions in the code, hardcoded logic, application specific integrated circuits (ASICs), processors, state machines, microcontrollers, and/or the like. The logic circuitry may also include memory circuits to store code and/or data, such as buffers, registers, random access memory modules, flash memory, and/or the like.

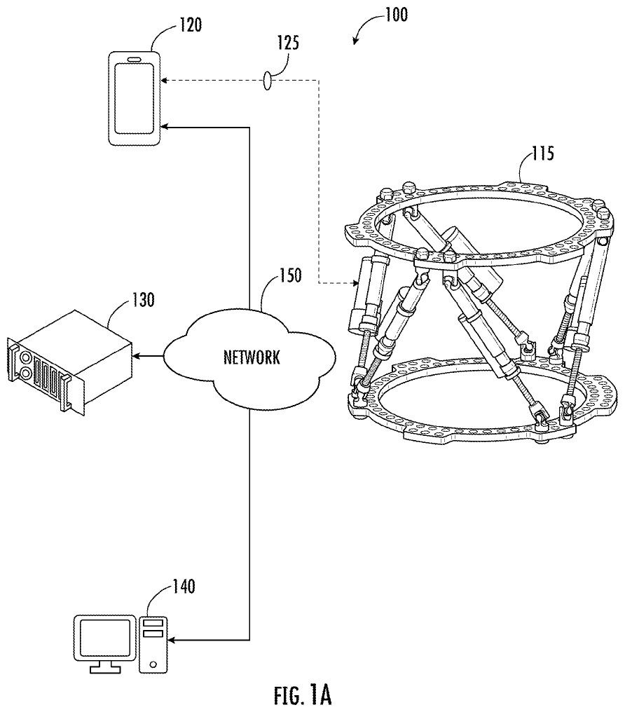

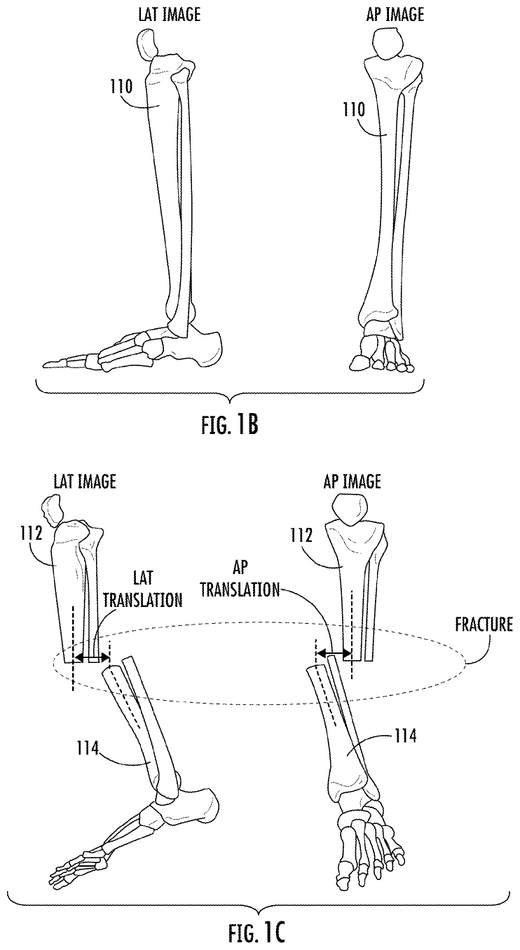

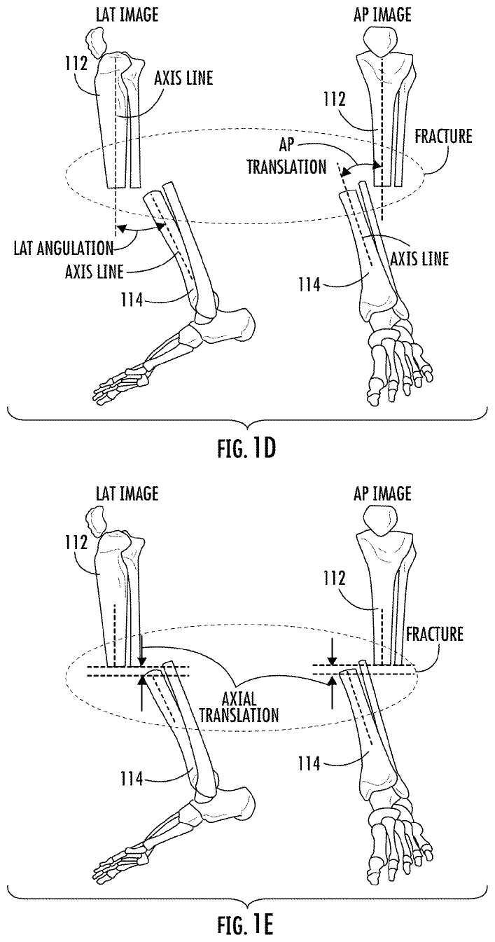

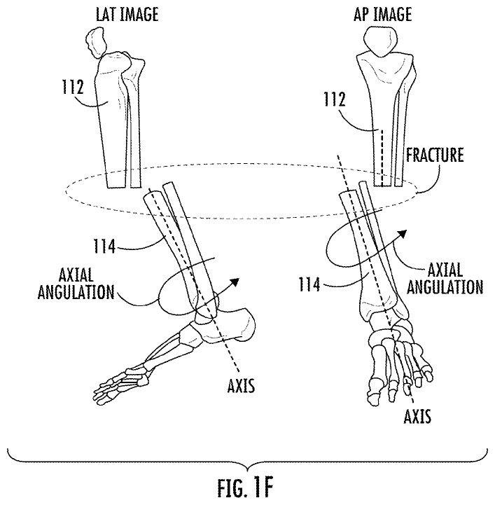

An example of a system 100 for treating a patient is illustrated in FIG. 1A. The system illustrated is only one example of a system that includes correction logic circuitry to generate a treatment plan including a prescription associated with micromotions for correction of a bone deformity of bone 110 (shown in FIG. 1B) with a bone alignment device 115. Other examples may use other types of orthopedic devices and/or processing circuitry to generate a treatment plan.

The system 100 may include the external bone alignment device 115 configured to couple to a patient, a patient device 120 connected to a network 150, a server 130 connected to the network 150, and a Health Care Practitioner (HCP) device 140 connected to the network 150. The illustrated external bone alignment device 115 may include, e.g., an automatic, six-axis external bone alignment device. In other examples, an external bone alignment device 115 may be any device capable of coupling to two or more bone segments of a bone 110 and moving or aligning the bone segments relative to one another.

The patient device 120 illustrated is a handheld wireless device. In other examples, a patient device may be any brand or type of electronic device capable of executing a computer program, outputting results to a patient, and communicating with wirelessly 125 or via wire with the external bone alignment device 115. For example, and without limitation, the patient device 120 may be a smartphone, a tablet, a mobile computer, or any other type of electronic device capable of providing one or both of input and output of information. In some examples, the patient device 120 may couple with the network 150 via wired and/or wireless connections to facilitate use of the patient device 120 to display, implement, and/or provide feedback related to implementation of a prescription for the external bone alignment device 115. In many examples, the server 130 and/or the HCP device 140 may transmit a prescription to the patient device 120 and/or updates for the prescription to the patient device 120 responsive to the feedback related to implementation of a prescription for the external bone alignment device 115.

In some examples, the patient device 120 may receive instructions for a set of micromotions to implement in conjunction with a prescription to perform a treatment plan for a patient. The set of micromotions may operate in conjunction with an app executing on the patient device 120 to instruct automatic components such as struts of the external bone alignment device 115 to perform adjustments in accordance with the adjustment schedule of the prescription as well as to perform micromotions during the treatment plan.

The instructions for the set of micromotions may include instructions related to the timing, rate, pattern, and/or rest interval for the set of micromotions. In some examples, the rate, timing, pattern, and/or rest intervals may be set by default preferences and/or user preferences in the app on the patient device 120. In some examples, the instructions may cause the patient device 120 to transmit commands via wireless communications to the external bone alignment device 115 at one or more portions of the treatment plan, in accordance with the instructions, such as prior to performing a first adjustment on the adjustment schedule, during an initial set of adjustments on the adjustment schedule, between adjustments on the adjustment schedule, during a middle set of adjustments on the adjustment schedule, during a final set of adjustments on the adjustment schedule, and/or after a final set of adjustments on the adjustment schedule.

In some examples, the commands may instruct processing circuitry of the external bone alignment device 115 to respond to the commands with feedback related to receipt and/or execution of commands for the micromotions. For instance, if a command instructs one or more automatic struts to adjust by, e.g., 0.1 mm or to adjust to a setting of, e.g., 2.1 mm, the external bone alignment device 115 may transmit a response and/or data as feedback responsive to the command. In some examples, the external bone alignment device 115 may transmit a response confirming receipt of the command. In some examples, the external bone alignment device 115 may transmit a response confirming execution of the command. In some examples, the external bone alignment device 115 may transmit a response including sensor data related to the execution of the command such as sensor data indicative of the magnitude of a physical adjustment of one or more automatic struts, sensor data indicative of an amperage applied to one or more motors of the automatic strut(s), sensor data related to the amount of force applied to adjust the one or more automatic strut(s), sensor data related to an amount of strain on one or more components of the external bone alignment device 115 such as on one or more automatic struts and/or on one or more of the rings of the external bone alignment device 115.

In many examples, the one or more sensors may be attached to and/or integrated with one or more components of the external bone alignment device 115. In some examples, the external bone alignment device 115 may include a single wireless communications interface, such as a transmitter and receiver or a transceiver, to receive commands from and send feedback to the patient device 120. In further examples, the external bone alignment device 115 may include more than one wireless communications interface. For instance, in some examples, external bone alignment device 115 may include a wireless communications interface coupled with each adjustment component such as each strut. In such examples, the sensors may couple with processing circuitry within one or more of the adjustment components and/or with processing circuitry coupled with the external bone alignment device 115.

In some examples, the patient device 120 may include a wireless communications interface for Bluetooth communications such as a wireless communications interface compatible with Bluetooth specifications such as Bluetooth Core Specification revision v5.3 published on Jul. 13, 2021, and/or Bluetooth Mesh Profile Specification revision v1.0 published on Jul. 13, 2017. The examples are not limited to these specifications.

In some examples, the patient device 120 may include a wireless communications interface for a wireless local area network (WLAN), such as a WLAN implementing one or more Institute of Electrical and Electronics Engineers (IEEE) 802.11 standards (sometimes collectively referred to as “Wi-Fi”). Such standards may include, for instance, the IEEE 802.11-2020, published Feb. 26, 2021, and the IEEE 802.11ax-2021, published Feb. 9, 2021. The examples are not limited to these standards.

In some examples, the patient device 120 may wirelessly communicate 125 via a Bluetooth protocol or a Wi-Fi protocol with the external bone alignment device 115 either directly or via another device such as an access point. In some examples, wherein the external bone alignment device 115 includes multiple adjustable components, the patient device 120 may transmit commands directly to more than one of or all the adjustable components individually to perform adjustments according to the adjustment schedule and/or to implement a set of micromotions. In such examples, each command may include an adjustment for the adjustable component to which the command is addressed.

In other examples, the patient device 120 may broadcast a command directly to more than one of or all the adjustable components to make adjustments according to the adjustment schedule and/or to implement a set of micromotions. In such examples, the command may include an element that identifies a series of one or more adjustments for one or more of the adjustable components. In some examples, the command may include a series of one or more adjustments and one or more delays that represent rest intervals.

In some examples, the patient device 120 may transmit commands to the adjustable components that include a rate, a rest interval, a pattern, and/or a magnitude for micromotions and processing circuitry on the external bone alignment device 115 may implement the micromotions accordingly. 125 The network 150 may be one or more interconnected networks, whether dedicated or distributed. Non-limiting examples include personal area networks (PANs), local area networks (LANs), wide area networks (WANs), metropolitan area networks (MANs), private and/or public intranets, the Internet, cellular data communications networks, switched telephonic networks or systems, and/or the like. Connections to the network 150 may be continuous or may be intermittent, only providing for a connection when requested by a sending or receiving client device.

The server 130 is shown connected to the network 150 in FIG. 1A. The server 130 may be a single computing device in some examples or may itself be a collection of two or more computing devices and/or two or more data storage devices that collectively function to process data as described herein. The server 130, or any one or more of its two or more computing devices, if applicable, may connect to the network 150 through one or both of firewall and web server software and may include one or more databases. If two or more computing devices or programs are used, the devices may interconnect through a back-end server application or may connect through separate connections to the network 150. The server 130 or any component server device of the system may include integrated or separate computer readable media containing instructions to be executed by the server. For example, and without limitation, computer readable media may be any volatile or non-volatile media integrated into the server 130 such as a hard disc drive, solid state drive, random access memory (RAM), and/or non-volatile memory such as flash memory. Such computer readable media, once loaded into the server 130 as defined herein, may be integrated, non-transitory data storage media. In some examples, the server 130 may include a storage location for information that will be eventually used by the patient device 120, the server 130, and/or the HCP device 140.

When stored on the server 130, memory devices of the server 130, as defined herein, provide non-transitory data storage and are computer readable media containing instructions. Similarly, computer readable media may be separable from the server 130, such as a flash drive, solid state drive, external hard disc drive, flash memory, tape drive, Compact Disc (CD), or Digital Versatile Disc (DVD) that is readable directly by the server 130 or in combination with a component connectable to the server 130.

In some examples, correction logic circuitry of the server 130 may communicate with the HCP device 140 via, e.g., a web browser or other client software installed on the HCP device 140 (correction logic circuitry). The correction logic circuitry may facilitate interaction with a user such as an orthopedic surgeon to create or correct a correction path for a bone alignment device such as the external bone alignment device 115. The correction logic circuitry may create or correct a correction path to correct a deformity of the bone 110 based on a set of one or more images such as radiographs, preoperative user input data (optionally), postoperative input data, user preferences, and data in data structures such as one or more databases or libraries.

In some examples, the correction logic circuitry of the server 130 may interact with the user graphically via the image(s) to create and/or adjust a correction path, divide the correction path into stages of correction, and/or the like. In other examples, the correction logic circuitry may reside on and may include, e.g., code for execution by a processor of the HCP device 140 so that a network 150 may not be required.

The correction logic circuitry may also determine one or more sets of micromotions to implement during a treatment plan to correct the deformity through interaction with the user and create instructions for the patient device 120 to implement the micromotions during the treatment plan. In many examples, the correction logic circuitry may provide a graphical user interface for the user to input information to describe the micromotions to implement including rest intervals between sets of the micromotions. For instance, the graphical user interface may include elements to allow the user to choose or enter a rate, a rest interval, a pattern, and a magnitude for micromotions via data entry, default settings, user preference settings, and/or the like. For instance, in some examples, the correction logic circuitry may include a default setting and/or user preference for a rate, a rest interval, a pattern, and/or a magnitude for each set of micromotions that the user adds to the treatment plan. The user may select the default rate or user preference rate or may select a different rate from a list of rates such as a pull-down menu. Similarly, the user may select the default or user preference rest interval, pattern, and/or magnitude or may select a different rest interval, pattern, and/or magnitude from lists such as pull-down menus.

In some examples, the server 130 may generate a set of instructions for the patient device 120 to implement the treatment plan. In some examples, the set of instructions may be specific to the manufacturer, model, and configuration of the patient device 120. In further examples, the correction logic circuitry may include an app installed on the patient device 120 and may configure the set of instructions for specific version of an app executing on the patient device 120. In still further examples, the correction logic circuitry may configure the set of instructions for one or more or all versions of the app that could be executing on the patient device 120. In some examples, the correction logic circuitry of the server 130 may configure the instructions for an app executing on the patient device 120 without need to factor in the manufacturer, model, and configuration of the patient device 120. In such examples, the app may act as an application programming interface (API) that is configured to execute a universal set of instructions to implement micromotions in conjunction with an adjustment schedule.

In some examples, the correction logic circuitry may include code executing on the HCP device 140 and on the server 130 and may include one or more databases operating on the server 130. The databases may include one or more data structures or libraries comprising multiple orthopedic devices for one or more different bones, fixations for the orthopedic devices, strut dimensions and adjustability, other hardware limitations/constraints, and/or the like. The databases may also include a library to generate instructions for micromotions with different patterns, rates, rest intervals, magnitudes, and/or the like. In several examples. The library may include several default sets of micromotion instructions. In some examples, the correction logic circuitry may interact with the user to adjust the default sets of micromotion instructions according to the user's preferences such as the patterns, rates, rest intervals, magnitudes, and/or the like.

In many examples, the correction logic circuitry may display a 2D or 3D model of the orthopedic device, such as the external bone alignment device 115, with the transosseous elements attached to the bone 110 and may provide for user interaction graphically or via keystrokes to create or adjust the correction path and the micromotions for the bone deformity. In some examples, the correction logic circuitry may also or alternatively interact with the user to create or adjust the micromotions associated with adjustments during the treatment plan.

In many examples, the correction logic circuitry may generate a graphical user interface with user interface elements that can be manipulated graphically or with keystrokes, to help the user determine a set of micromotions to associate with waypoints of the correction path, or adjustments on the adjustment schedule. The user may interact with the user interface elements, e.g., to adjust the perspective view of the bone 110 in a 3D image, to adjust the point in time (or day of micromotions and/or adjustment) of a prescription illustrated by the image(s), to display a projection of a 3D curve of the correction path on a 3D image or on two or more 2D images, to display differences in deformity correction between the current adjustment and a new adjustment, and/or the like. In some examples, the correction logic circuitry may illustrate a current correction path and a revised correction path, based on interaction with a user, as points and/or curves on the image(s) of the bone 110.

In some examples, the graphical user interface of the correction logic circuitry may present a series of images to illustrate performance of the micromotions. For instance, after a user determines a set of micromotions associated with one or more adjustments or waypoints, the graphical user interface may illustrate, with a series of 2D images or via a 3D model or image, the movement of the moving bone segment in relation to the static bone segment during the performance of the micromotions.

In some examples, the correction logic circuitry may include user interface elements to interact with a user to show the progression of movement of the moving bone segment including sets of micromotions throughout a correction path of a prescription to the final corrected state of the bone 110. In some examples, the user may play the correction path of a prescription forward to show the progression along the correction path of the moving bone segment from the initial postoperative state to the final corrected state, illustrating the timing of the adjustments along the correction path and, optionally, the micromotions defined by the user or set by default preferences or via user settings throughout the treatment plan. In further examples, the user may also play the progression of the correction path for the prescription in reverse from the final corrected state to the initial postoperative state including the adjustments and optionally the micromotions.

In some examples, the correction logic circuitry may perform an impingement analysis based on one or more correction paths for the struts, distal ring(s), transosseous elements coupled with the distal ring(s) and fixations. In some examples, the correction logic circuitry may determine movements required to achieve the final corrected state based on postoperative user inputs of the initial bone deformity and the final corrected state of the bone 110. In other examples, the correction logic circuitry may use a current prescription and/or a user modified prescription to perform impingement analyses for the external bone alignment device 115.

Note that examples can use images captured from any angle or orientation and movements of bone segments may be defined in relation to the coordinate system implemented by the correction logic circuitry. Thus, references to vertical or horizontal movements relative to a 2D or 3D image may not reflect the actual components of such movements determined and stored by the correction logic circuitry unless properly oriented by the user. For instance, a vertical movement with respect to a particular image may represent movement along an x-axis, a y-axis, a z-axis, or any combination thereof, with respect to the coordinate system implemented by the correction logic circuitry. Thus, the correction logic circuitry may record such movements as a tuple or vector such as (x, y, z), where x, y, and z represent numbers indicative of movement in units such as millimeters or centimeters along the x-axis, y-axis, and z-axis, respectively. A movement of zero, in some examples, may represent no movement, a negative movement may represent movement in a first direction with respect to an axis, and a positive movement may represent movement in a second direction with respect to the axis.

AP and LAT views are common practice for radiographs of fractures and bone deformities, but examples are not limited to AP and LAT view images. Furthermore, as long as each of the images has a known scale, the images do have to be the same scale. The correction logic circuitry may translate or convert scales to a selected or default scale implemented by the correction logic circuitry and translate or convert movements associated with bone segments and struts in images to a coordinate system implemented by the correction logic circuitry.

Note that examples are not limited to the correction logic circuitry residing in the server 130. The correction logic circuitry may reside in whole or in part in the HCP device 140. The correction logic circuitry may reside in whole or in part in the patient device 120. The correction logic circuitry may reside in whole or in part in the server 130. Furthermore, the correction logic circuitry may reside partially in multiple compute servers and data storage servers managed by a management device and operating as the server 130. The correction logic circuitry may also or alternatively reside partially in multiple computers and/or storage devices such as the HCP device 140 and the patient device 120. Where the correction logic circuitry may reside partially in multiple computers, the correction logic circuitry may include management logic circuitry to manage multiple local and/or remote resources.

The HCP device 140 is shown connected to the network 150. The HCP device 140 illustrated is a desktop personal computer. In other examples, the HCP device 140 may be any brand or type of electronic device capable of executing a computer program and receiving inputs from or outputting information to a user. For example, and without limitation, the HCP device 140 may be a smartphone, a tablet computer, or any other type of electronic device capable of providing one or both of input and output of information. Such a device may provide a user interface for data input, waypoint or code block modification, determination of one or more sets of micromotions to associate with an adjustment schedule or prescription, generation of instructions for the one or more sets of micromotions, as well as communication with the patient device 120, another HCP, or a device or system manufacturer.

An HCP device such as the HCP device 140 may be connected to the network 150 by any effective wired or wireless communications interface. For example, and without limitation, the connection may be by wired and/or wireless connection through any number of routers and switches. Data may be transmitted by any effective data transmission protocol. The HCP device 140 may include integrated or separate computer readable media containing instructions to be executed by the HCP device 140. For example, and without limitation, computer readable media may be any media integrated into the HCP device 140 such as a hard disc drive, solid state drive, RAM, or non-volatile flash memory. Such computer readable media once loaded into the HCP device 140 as defined herein may be integrated and non-transitory data storage media. Similarly, computer readable media may be generally separable from the HCP device 140, such as a flash drive, solid state drive, external hard disc drive, CD, or DVD that is readable directly by the HCP device 140 or in combination with a component connectable to the HCP device 140.

In some examples a surgeon user will utilize the correction logic circuitry by means of any combination of the patient's device 120, a server 130, or a HCP device 140 to generate an adjustment schedule or prescription with planned micromotions. The adjustment schedule or prescription and any planned micromotions are then transmitted to external bone alignment device 115 from generating device. Communication of the prescription from the correction logic circuitry to the external bone alignment device 115 is not required to utilize a patient device 120. Circuitry on the external bone alignment device 115 including any combination of processors, memory, circuitry, wireless network interfaces, or wired interfaces may enable the external bone alignment device 115 to store and follow the instructions of the adjustment schedule or prescription including any planned micromotions without further instructions from the patient's device 120, a server 130, or a HCP device 140. The external bone alignment device 115 may communicate data wirelessly 125 or via wire to patient device 120 which will communicate the data to the other devices on a network 150. In some embodiments the external bone alignment device 115 may communicate data wirelessly or via wire directly server 130, HCP device 140, or network 150.

FIGS. 1B-1F illustrate LAT and AP images of an unfractured tibia, bone 110, and the same tibia fractured into a first bone segment 112 and a second bone segment 114. Each of FIGS. 1C-1F illustrate at least one of the deformity parameters on the LAT image and the AP image. Note that while the illustrations focus on the tibia and LAT and AP images, examples may process any other bone and any other viewing angle in a similar manner.

FIG. 1B illustrates an example of an AP and a LAT image of an unfractured tibia, bone 110. Note that the AP image provides a fontal view of the tibia and the LAT view provides a side view of the tibia.

FIG. 1C illustrates an example of the tibia bone 110 fractured into two bone segments, a first bone segment 112 and a second bone segment 114. As discussed therein, the first bone segment typically refers to the fixed bone segment if the processing involves a fixed bone segment. For instance, some examples fix the first bone segment, and all deformity parameters are determined based upon movement of the second (moving) bone segment to align the second bone segment with the first bone segment.

In FIG. 1C, the example may determine the LAT translation based on a horizontal translation of the second bone segment 114 to align the second bone segment with the first bone segment 112 on the LAT image. Similarly, the example may determine the AP translation based on a horizontal translation of the second bone segment 114 to align the second bone segment with the first bone segment 112 on the AP image.

FIG. 1D illustrates an example of the tibia bone 110 divided into two bone segments, a first bone segment 112 and a second bone segment 114 for purpose of illustrating the deformity parameters of LAT angulation and AP angulation. A way to illustrate and/or determine the LAT or AP angulation is to overlay a first axis line through the axis of the first bone segment 112, overlay a second axis line through the axis of the second bone segment 114, and measure the angle between the first and second axis lines. The angle between the first and second axis lines may be the LAT or AP angulation, depending on the view.

FIG. 1E illustrates an example of the tibia bone 110 fractured into two bone segments, a first bone segment 112 and a second bone segment 114 for purpose of illustrating the deformity parameter of axial translation. Many examples determine the axial translation as the vertical movement of either or both the first bone segment 112 and the second bone segment 114 to bring the two bone segments together. Many examples determine the final axial translation based on interaction with the user. For 2D deformity parameters, the final axial translation may be determined from a single image. For 3D deformity parameters, the final axial translation parameter may be determined after calculation of an axial translation for two or more images such as a LAT view and an AP view of the bone segments. Some examples may have a user define an origin one point on one bone segment and a corresponding point on the other bone segment such that translation may be defined as the component distances between the origin and corresponding points.

FIG. 1F illustrates an example of the tibia bone 110 fractured into two bone segments, a first bone segment 112 and a second bone segment 114 for purpose of illustrating the deformity parameter of axial angulation. The axial angulation is the rotation of the second bone segment 114 about the axis of the second bone segment 114 to align the second bone segment with the first bone segment 112. In many examples, the axial angulation is determined clinically.

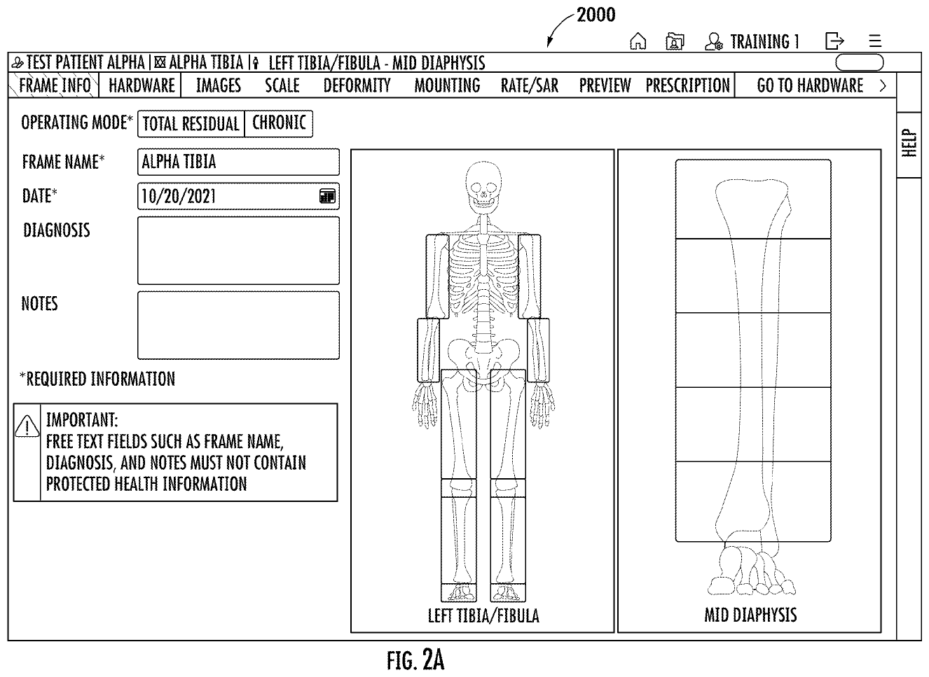

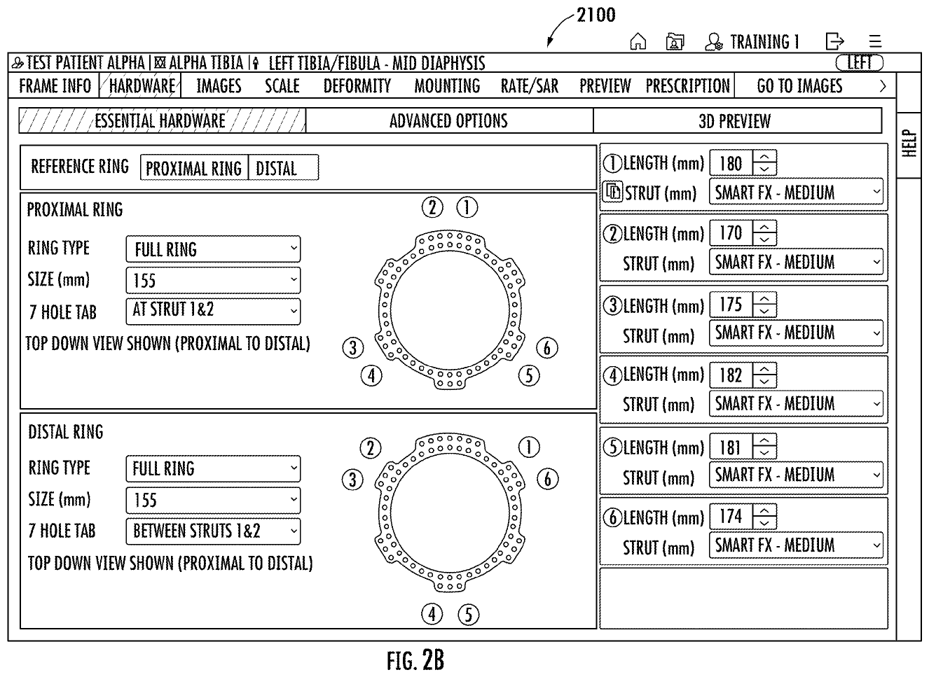

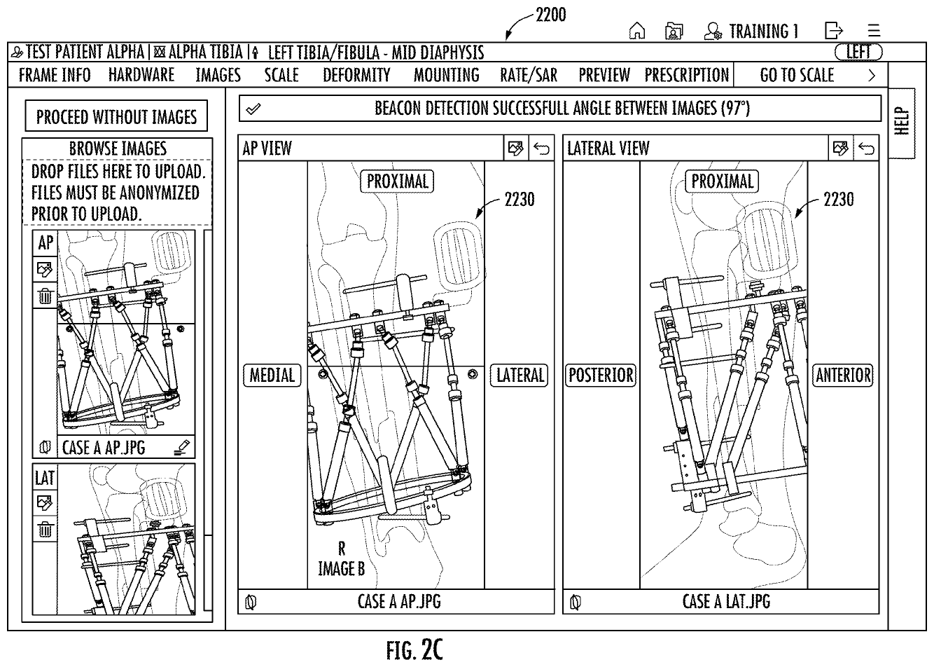

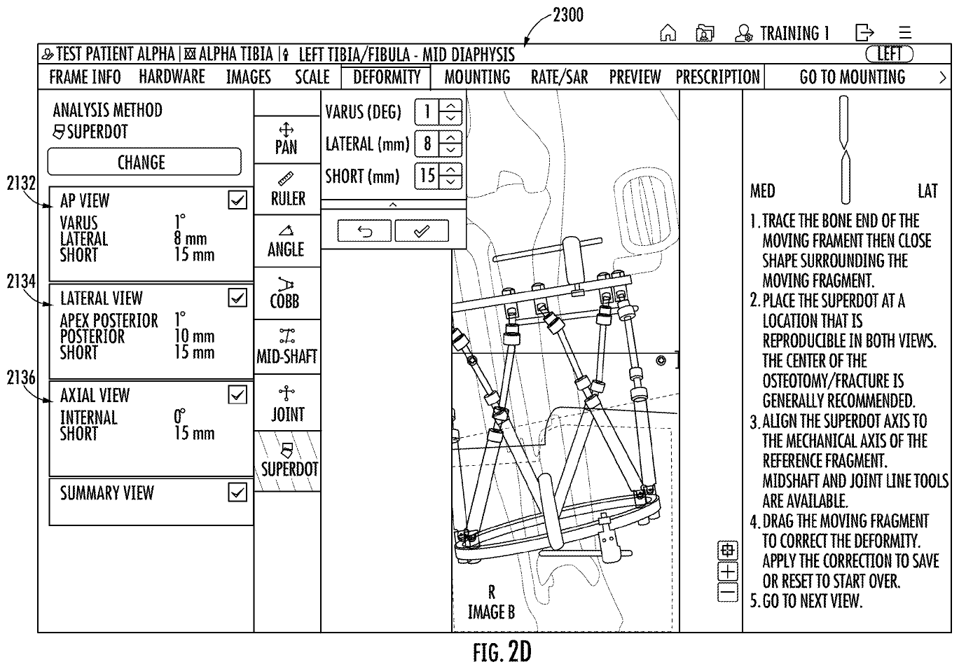

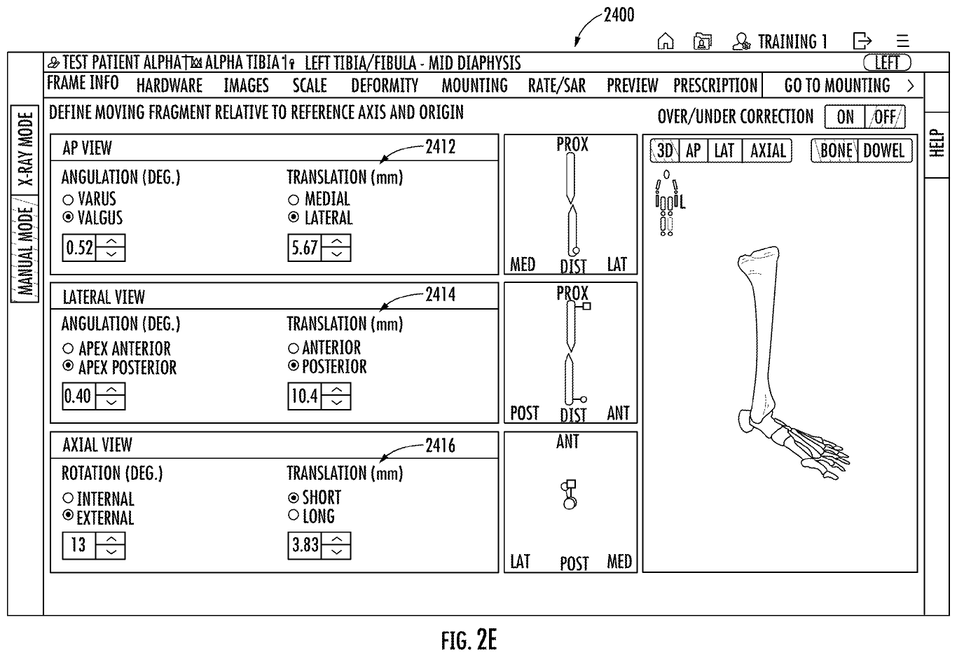

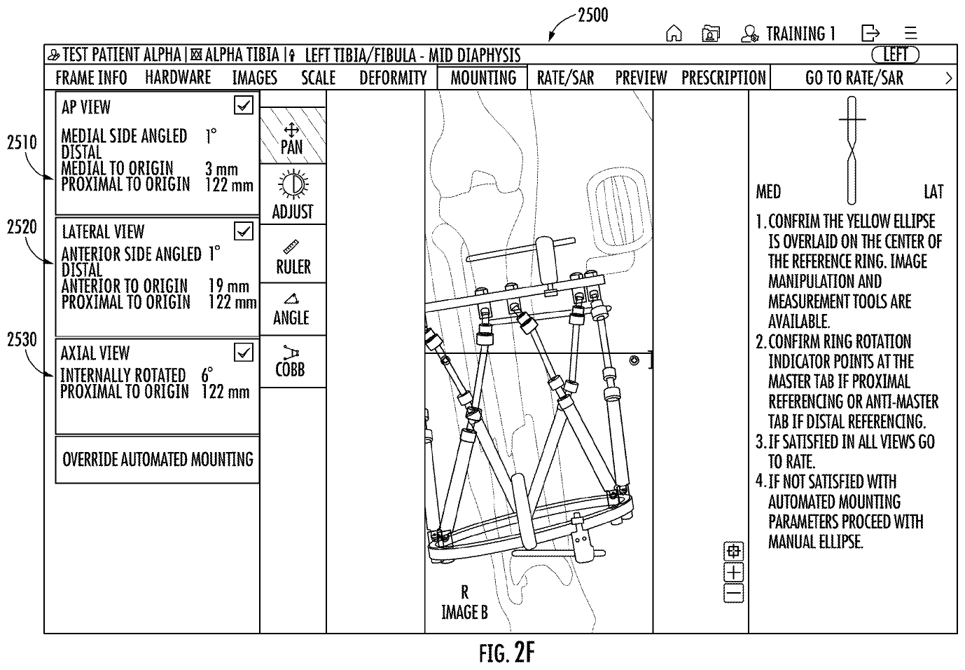

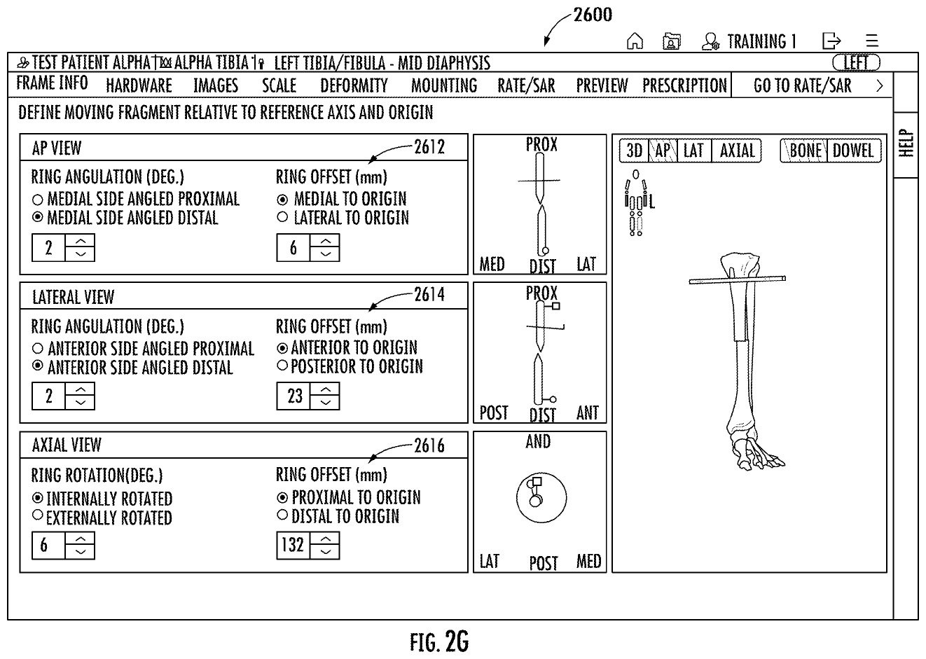

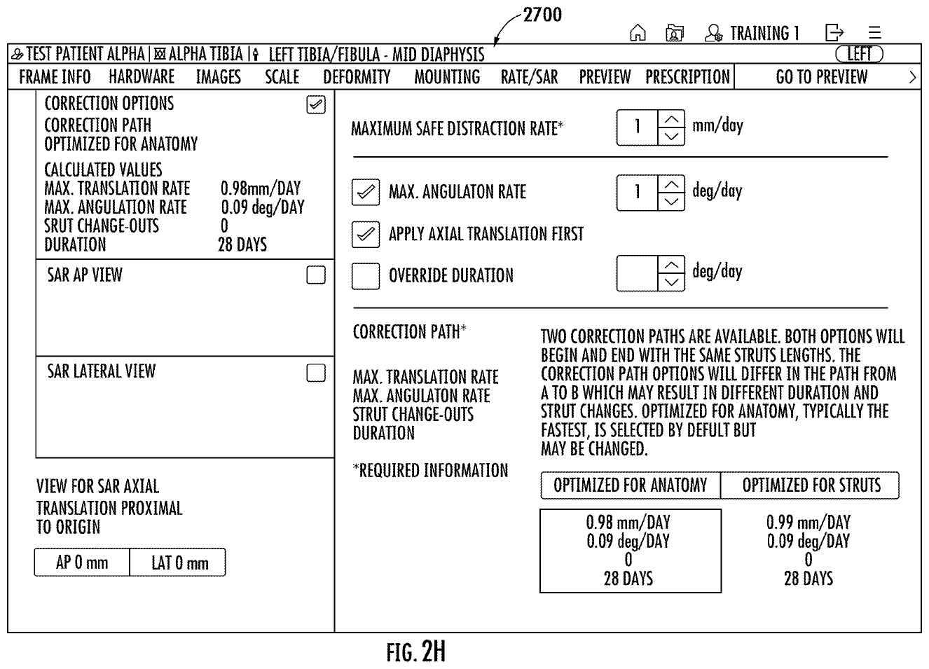

FIGS. 2A-H illustrate examples of a user interface to input data to correction logic circuitry such as the correction logic circuitry discussed in conjunction with FIGS. 1A-F.

FIG. 2A illustrates an example of a user interface 2000 of correction logic circuitry for user input data comprising information such as a file name, diagnosis, notes, general anatomical region of interest, and anatomical hand.

FIG. 2B illustrates an example of a user interface 2100 of correction logic circuitry for a user to input data about the bone alignment device. In some examples, the user input data includes identification of the reference hardware component, such as a reference ring. In some examples the selection of a reference hardware component may determine the fixed and moving bone segments. The user input data may also include identification relevant hardware parameters such as for, e.g., the ring type, ring size, strut lengths, strut sizes, strut types, strut mount locations. In other examples, the number and/or the types of hardware may differ depending on the type of bone alignment device. In other examples, the user interface 2100 may also include additional data entry for the hardware such as the type, size, location, angle, and mounting hardware for the transosseous elements that attach the bone alignment device to the bone segments. Such examples may include impingement analysis that includes transosseous elements, neurovascular structures, mounting hardware for the transosseous elements, and/or the like in addition to the struts and rings. Such examples may also include a database such as an electronic library of hardware components and dimensions such as the dimensions of the struts, rings, transosseous elements, mounting hardware for the transosseous elements, and/or the like.

In some examples, the edge geometry of the bone alignment device is defined to allow for impingement analysis. In some examples, edge geometry is defined when the user selects the hardware components from a list via a data structure such as a library. In other examples, edge geometry must be input manually or defined on medical images (e.g., AP and Lateral radiographs) for relevant hardware components instead of or in addition to selection from a list. In some examples the edge geometry of hardware components may be defined in medical images automatically by the correction logic circuitry via edge detection algorithms, radiographic markers, and the like.

FIG. 2C illustrates an example of a user interface 2200 for correction logic circuitry to upload images for the bone deformity such as 2D AP and LAT radiological images. Note that, in some examples, the correction logic circuitry may allow the user to choose to upload and scale radiological images if radiological planning is desired or may allow the user to proceed without uploading images to define deformity parameters manually. The radiological planning may allow the user to identify the deformity and mounting parameters via graphical interaction with the radiological images.

FIG. 2D-E illustrate examples of a user interface 2300 or 2400 for the correction logic circuitry to obtain input data about the bone deformity.

FIG. 2D illustrates the user interface 2300 may allow the user to graphically identify the bone deformity in the AP view 2312, the Lateral view 2314, and the Axial view 2316 via the medical images (e.g., AP view and LAT view radiographs), and/or to manually enter the deformity.

FIG. 2E illustrates an example of a user interface 2400 of the correction logic circuitry that offers manual entry of the deformity parameters as an alternative to entry graphically via the radiological images. In the example the deformity parameters are manually defined in the AP view 2412, Lateral View 2414, and Axial View 2416.

The method of analyzing the deformity is unimportant if the deformity parameters can be related back to points on the bone segments and/or hardware. In some examples, like the examples shown inf FIG. 2D-E, the deformity parameters will be input as 2D components of the 3D deformity parameters (e.g., AP angulation, AP translation, LAT angulation, LAT translation, Axial angulation (rotation), and Axial translation). Other examples may directly capture or allow input of the 3D deformity parameters.

FIG. 2F-G illustrate examples of a user interface 2500 or 2600 for the correction logic circuitry to obtain input data about the location of the bone alignment device hardware relative to the bone segments. In some examples, a point on the bone alignment device is described relative to a point on the bone segments.

FIG. 2F illustrates an example of a user interface 2500 of the correction logic circuitry to facilitate graphical data entry of information about the bone alignment device such as an external bone alignment device. The correction logic circuitry may use image analysis presented in the user interface 2500 to graphically identify location the bone alignment device hardware in the AP view 2510, the Lateral view 2520, and the Axial view 2530 via medical images (e.g., AP view and LAT view radiographs) or data inputs. In some examples, the correction logic circuitry may calculate the bone alignment device location data automatically from the medical images via edge detection algorithms, radiographic markers, and the like.

FIG. 2G illustrates an example of a user interface 2600 of the correction logic circuitry that offers manual data entry of the hardware parameters as an alternative to automated data entry of hardware parameters based on image analysis of the bone alignment device via the radiological images. In some examples, the user interface 2600 provides a user interface element (not shown) such as a manual mode button and an x-ray mode button to select the method of entry of the hardware parameter input data. In the present example, the manual mode button of the user interface element may be selected to select the user interface 2600 rather than a user interface 2500 as shown in FIG. 2F.

In some examples a single reference hardware component such as a ring is described relative to the bone segments. In the example of 2600, the mounting parameters are manually defined in the AP view 2612, Lateral View 2614, and Axial View 2616. The locations of additional hardware components of the bone alignment device may be automatically defined by the means of connection to the referenced hardware component (e.g., the location of a second ring may be defined by the struts connecting the second ring to a reference ring). In some examples the connection constraints of specific hardware components are defined when they selected by the user via a data structure such as a library. In some examples, the user may directly input the location of all relevant hardware components into the correction logic circuitry.

FIG. 2H illustrates an example of a user interface 2700 of the correction logic circuitry that offers entry of rate limiting parameters for a prescription including a maximum safe distraction rate in millimeters per day and a maximum angulation (rotation) rate in degrees per day. Rate limiting parameters are used to control rate at which bone segments are adjusted and to calculate the duration of a prescription.

FIG. 2I illustrates an example of a prescription 2800 with daily adjustments. The prescription is the ultimate output of the correction logic circuitry. Note that, in the presented example, the prescription has two adjustment stages. Although not shown in FIG. 2A-H, a GUI may be present in the correction logic circuitry to define multiple adjustment stages with independent rates, durations, and correction paths. Prescriptions with multiple adjustment stages will result in the bone segments traveling from one waypoint to another along the correction path of the prescription until all prescribed adjustment stages are complete. Prescriptions are not limited to one adjustment of each bone alignment device component per day.

Each adjustment, which coincides with each day of the prescription in the present example, describes the settings (length of the strut) for each of the struts. The difference between the current setting of a strut and the subsequent setting of the strut is referred to as the adjustment, which may be represented with a unit of length such as millimeters (mm). With an automated bone alignment device, adjustments may occur through each day and with small adjustment resolutions that are not detailed within the daily adjustment schedule. Each set of adjustments whether daily or within a shorter time defines the location and orientation of the moving bone segment along the correction path of the prescription. The location and orientation of the moving bone segment after a set of adjustments can be referred to as a waypoint of the correction path. It is possible to have more than one waypoint along the correction path within each day of adjustment. In the present example, the prescription presents the length of the strut for each of the six struts of a bone alignment device. In other examples, the prescription may describe the adjustments to the length of the struts in addition to or in lieu of the length of the struts. For instance, strut 1 includes a strut length at day 0 of 180.00 mm and a strut length at day 1 of 181.00 mm. Thus, the length of the strut 1 is increased by 1 mm as part of the adjustment on day 1. The prescription may show the adjustment of 1 mm for day 1, 1 revolution of strut 1, 1 click of strut 1, or the like in addition to the overall length of the strut 1 or as an alternative to showing the overall length of the strut 1.

In the present example, the prescription is for an automated adjustment components such as automated struts so the granularity of movement of a motor and gear ratio(s) between the axle (or shaft) of the motor and an adjustment mechanism of the strut may determine the granularity of the movements of the struts. For instance, if the gear ratio between the motor axle and the adjustment nut of the strut is 1:4 meaning that the gear attached to the motor's axle has ¼^{th} the teeth as the gear attached to the strut adjustment mechanism, one turn of the motor axle only turns the shaft of the strut by a quarter of a turn. In one turn of the strut's shaft is a 1 mm adjustment to the length of the strut, one turn of the motor axle is a 0.25 mm adjustment to the length of the strut.

Before, during, and/or after generation of the prescription, the correction logic circuitry of the server 130, the HCP device 140, or the patient device 120 may interact with the user to determine micromotions for the treatment plan and generate instructions based on the micromotions for execution by external bone alignment device 115. In many examples, the instructions may be associated with the prescription and transmitted to the external bone alignment device 115 for implementation.

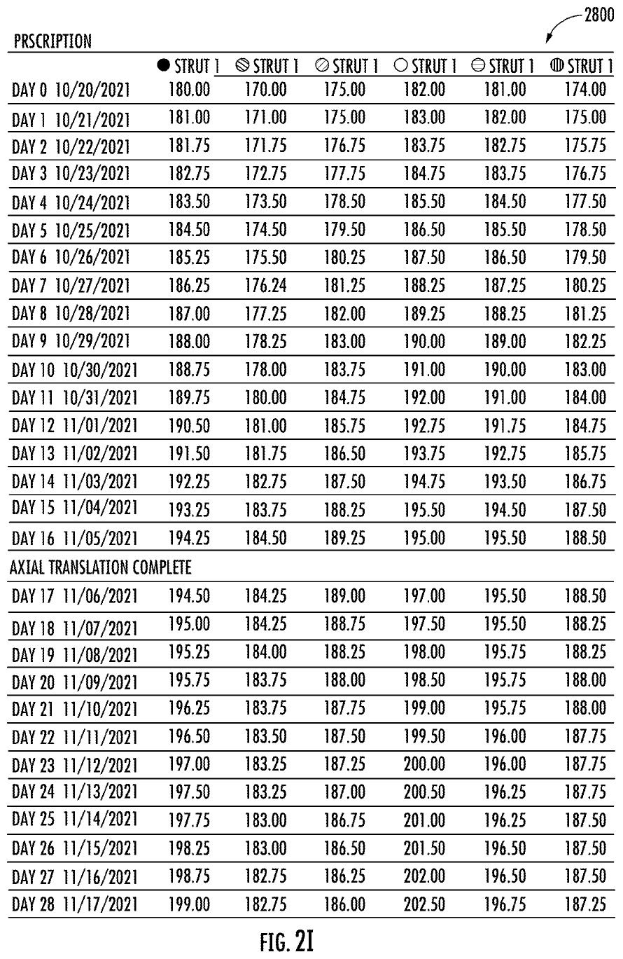

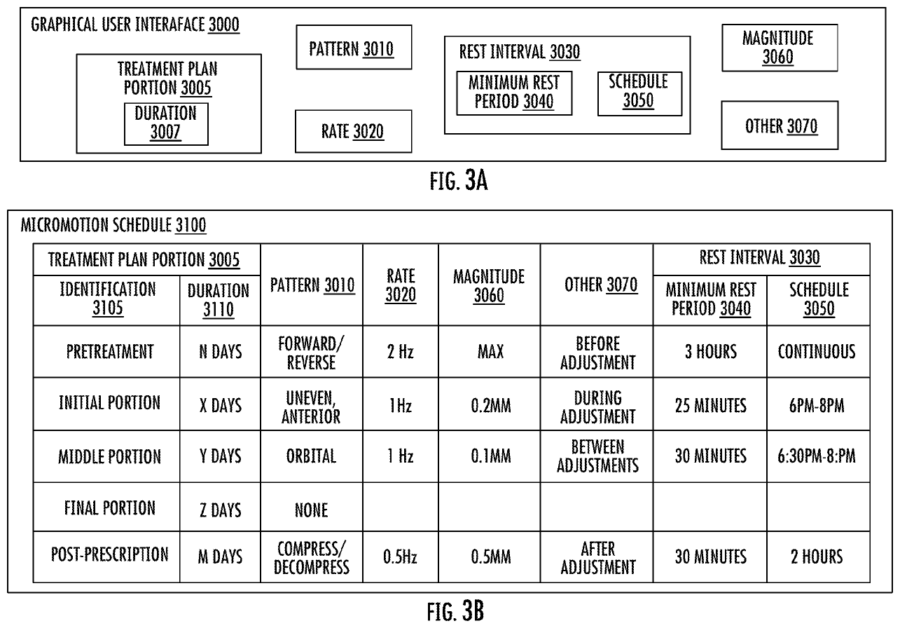

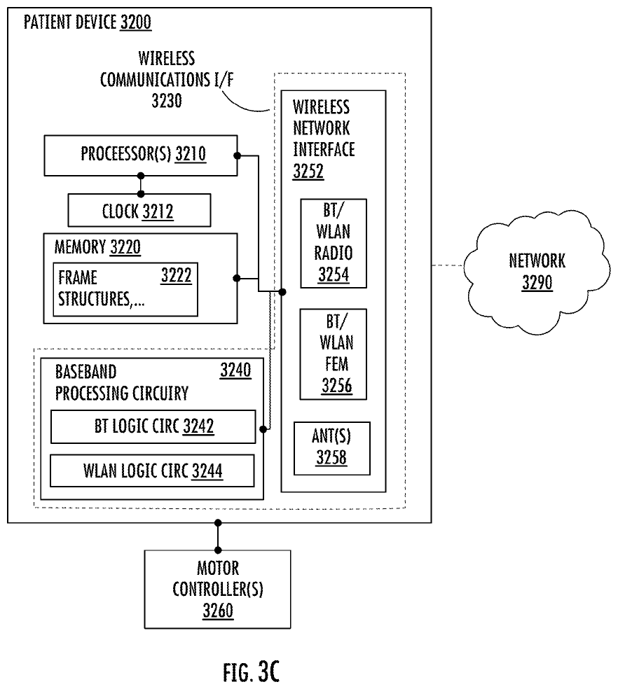

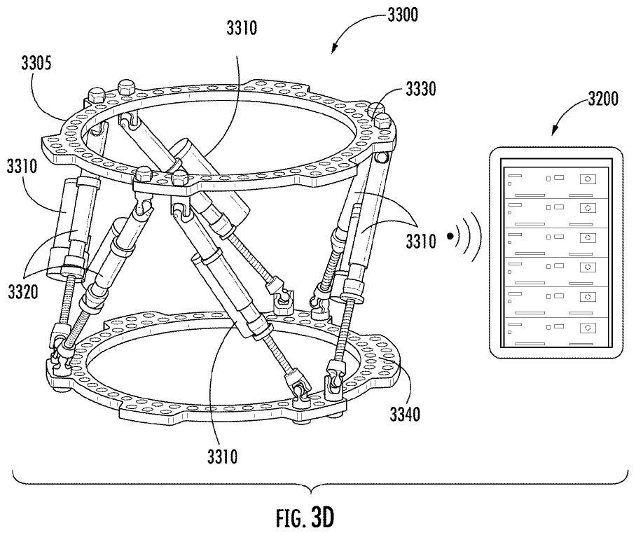

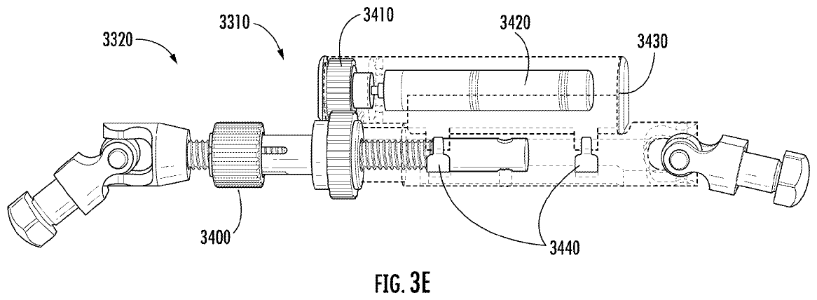

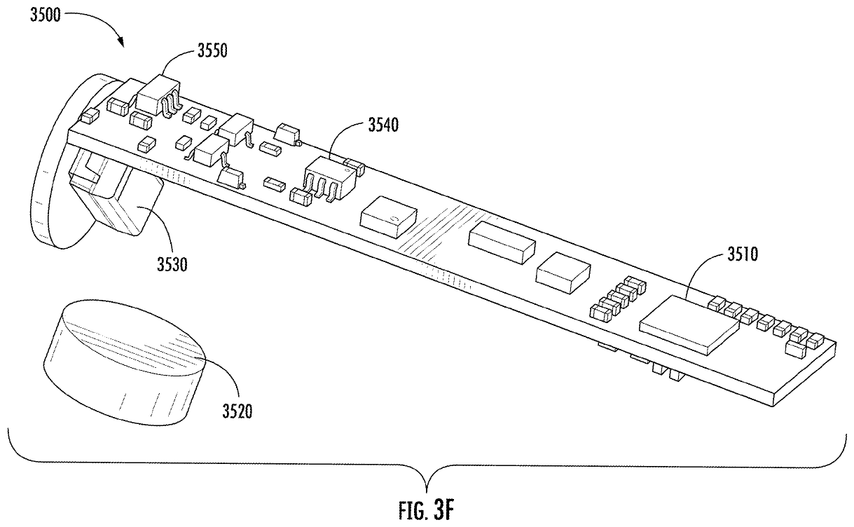

FIG. 3A illustrates an example of a graphical user interface (GUI) 3000 of correction logic circuitry such as the correction logic circuitry discussed in conjunction with FIG. 1A to determine micromotions to associate with a prescription such as the prescription 2800 shown in FIG. 2I. FIG. 3B illustrates an example of a micromotion schedule 3100 associated with a prescription such as the prescription shown in FIG. 2I. The micromotion schedule includes information gathered from a user and/or from default settings or user preference settings related to micromotions to perform in conjunction with the prescription.

The graphical user interface 3000 may display on the server 130, the HCP device 140, and/or the patient device 120 to interact with a user to determine micromotions to associate with a treatment plan for the prescription. The GUI 3000 may include one or more user interface elements such as a treatment plan portion 3005, a pattern 3010, a rate 3020, a rate interval 3030, a magnitude 3060, and/or one or more other 3070 user interface elements. The treatment plan portion 3005 may include a list or the like of selectable entries to identify the portion of the treatment plan for the prescription during which to perform micromotions. For example, the user may select a pretreatment portion, initial portion, middle portion, final portion, a post-prescription portion, and/or an entire treatment plan duration as illustrated in the micromotion schedule 3100 shown in FIG. 3B. The user may also select different micromotion options to occur during specific adjustment phases of a prescription. For example, a user may specify selected micromotions to begin when a specified waypoint of the prescription is reached and end when another defined waypoint is reached.

The treatment plan portion 3005 may automatically suggest a number of days and/or a percentage of the prescription for each of the selectable portions of the treatment plan and insert the number of days and/or a percentage in a duration 3007 user interface element field. In many examples, the user may edit the value in the duration 3007 user interface element. The micromotion schedule 3100 includes an example of the information or data collected from the user to define the micromotions in columns under the user interface element field labels such a treatment portion 3005, pattern 3010, rate 3020, magnitude 3060, other 3070, and rest interval 3030. The treatment plan portion 3005 may gather data for one or more of the portions selected by the user including an identification 3105 of the portion of the treatment plan and a value for the duration 3110 of the treatment plan portion. In the present example, the identification includes a description. In other examples, the treatment portion may be identified via a portion identifier such as a number, character, alphanumeric number, acronym, abbreviation, and/or the like.

In some examples, each portion may be associated, by default or by user preference settings, with a fixed duration or a percentage of the overall duration of the prescription. For instance, if the prescription is 28 days long, the pretreatment portion may be 1 day as a default fixed setting. The initial portion and middle portion may each include ⅓rd of the duration of the prescription such as 9 days. The final portion of the prescription may include the remainder of the duration of the prescription such as 10 days and the post-treatment portion may include a fixed duration such as 1 day.

In many examples, the user may also set the number of days or the percentage of days representing each of the portions of the treatment plan as a user preference. In such examples, the user may adjust the values (number of days or percentage) for the current prescription in the duration 3007 user interface element. For instance, the GUI 3000 may present the default values for each of the portions for the prescription in response to the user selecting the entire treatment plan for the treatment plan portion 3005. The correction logic circuitry may fill in values for the micromotions based on default values or as user preference settings to create a micromotion schedule such as the micromotion schedule 3100 shown in FIG. 3B. Thereafter, the user may edit each of the settings.

The GUI 3000 may provide a selectable list or the like for the pattern 3010 user element including each predefined pattern of micromotions that the user may select. The pattern 3005 may define two or more directions that the micromotions travel. In some examples, the user may create a custom pattern via the pattern 3010 user interface element.

The pattern 3010 column in the micromotion schedule 3100 includes examples of some of the patterns that the user may choose including a forward/reverse pattern, an uneven pattern, an orbital pattern, and a compress/decompress pattern. The forward/reverse pattern may include a pattern of micromotions to move the moving bone segment a magnitude such as 0.2 mm in a forward direction along the correction path and then move the moving bone segment a magnitude such as 0.2 mm in a reverse direction along the correction path to return the moving bone segment to the starting point of the micromotion.

The uneven pattern may move the moving bone segment in a pattern that unevenly applies a force and/or strain to a side of the static bone segment and a side of the moving bone segment to stimulate bone growth. In the present example, the user may select the uneven pattern as well as the side such as anterior, posterior, medial, and/or lateral.

The orbital pattern may move the moving bone segment in an orbital pattern about one or more points along the correction path. For instance, in some examples, the orbital path may orbit around the current position of a point on the moving bone segment such as when the micromotions are applied between adjustments on the adjustment schedule. In some examples, the orbital path may orbit around points along the correction as the moving bone segment is adjusted from a first waypoint on the correction path to a second way point on the correction path such as when the micromotions are applied during adjustments on the adjustment schedule.

The compress/decompress pattern may move the moving bone segment forward, along the mechanical axis of the static bone segment, compressing the moving bone segment into the static bone segment by a magnitude 3060, e.g., 0.05 mm. Thereafter, the compress/decompress pattern may move the moving bone segment in reverse, along the mechanical axis of the static bone segment, decompressing the moving bone segment by the magnitude 3060, e.g., 0.05 mm.

The rate 3020 user interface element may interact with the user by providing a list or a data entry field to set a value for the rate of performance of the micromotions to a rate between, e.g., 0.5 Hz and 3 Hz. In some examples, for a pattern that includes movement in two directions, one cycle may include movement in both directions such as forward and reverse. In some examples, one cycle may include movement in one direction such as forward or reverse. In some examples, one cycle may include one or more movements through an entire pattern of a micromotion.

The rest interval 3030 user interface element may interact with the user by providing a list or a data entry field to set a value for a minimum rest period and may optionally or alternatively offer the user an opportunity to set a schedule for performance of the micromotions. In some examples, the user may indicate a rest period such as a time period between 10 minutes and 8 hours. In some examples, the user may also or alternatively select days of the adjustment schedule and time periods within which to perform the micromotions.

To illustrate, the micromotion schedule 3100 identifies different sets of micromotions for each portion of the treatment plan. For the pretreatment portion, the rest period 3040 value is set to 3 hours and the schedule 3050 is set to continuous. In other words, during the duration 3110 of N days of the pretreatment portion of the treatment plan, the correction logic circuitry of the patient device will continuously perform forward/reverse micromotions with 3-hour rest periods.

For the initial portion, the rest period 3040 value is set to 25 minutes and the schedule 3050 is set to 6 pm to 8 pm. For the middle portion, the rest period 3040 value is set to 25 minutes and the schedule 3050 is set to 6:30 pm to 8 pm. For the final portion, the user determined not to perform micromotions. And, for the post-prescription portion, the rest period 3040 value is set to 30 minutes and the schedule 3050 is set to 2 hours.

The magnitude 3060 user interface element may interact with the user by providing a list or a data entry field to set a value for the magnitude of performance of the micromotions to a rate between, e.g., zero and maximum. For instance, the micromotion magnitudes may range between 0.05 mm and 0.5 mm. In some examples, the user may set the magnitude 3060 to minimum, which may include the smallest incremental movement possible with the automatic bone alignment device. In some examples, the user may set the magnitude 3060 to maximum, which may include the largest incremental movement possible within the boundaries of the correction path. In further examples, the user may select a magnitude such as 0.05 mm, 0.1 mm, 0.2 mm, or the like.

The other 3060 user interface element may interact with the user by providing a list or a data entry field to set a value for the other 3060 user element. In some examples, the user may set the other 3060 to an indication about the timing of the performance of the micromotions. For instance, the user may set the timing for performance of the micromotions to a time relative to adjustments on the adjustment schedule.

In the present example, the user may identify the timing for performance of the micromotions as before an adjustment, during an adjustment, after an adjustment, or between adjustments. For example, performance of micromotions before an adjustment may indicate that the micromotions should be allowed to complete prior to initiating an adjustment that moves the moving bone segment from a first waypoint to a subsequent waypoint on a correction path. Performance of micromotions during an adjustment may indicate that the micromotions should be performed during an adjustment that moves the moving bone segment from a first waypoint to a subsequent waypoint on a correction path. Performance of micromotions between adjustments may indicate that the micromotions should be performed after an adjustment and before a subsequent adjustment. Performance of micromotions after an adjustment may indicate that the micromotions should be performed after an adjustment. Many examples may require a rest period 3040 between an adjustment and performance of the micromotions.

FIG. 3C illustrates an example of a patient device 3200 such as the patient device 120 illustrated in FIG. 1A. The patient device 3200 may receive one or more frames such as data frames including a prescription with instructions for micromotions for a patient via a network 3290. For instance, the one or more frames may include medium access control (MAC) layer frames that are included within one or more physical layer frames often referred to as physical layer (PHY) data units (PPDUs) in Wi-Fi networks. In some examples, a MAC layer frame may include a header, a frame body, and a footer such as a frame check sequence (FCS). A PPDU may include a physical layer preamble, header, and data that includes one or more MAC frames.

The patient device 3200 may include processor(s) 3210 and memory 3220. The processor(s) 3210 and/or memory 3220 may include correction logic circuitry to cause an automatic bone alignment device to perform micromotions in conjunction with adjustments from an adjustment schedule of a prescription. The processor(s) 3210 may include any data processing device such as a microprocessor, a microcontroller, a state machine, processing circuitry, and/or the like, and may execute instructions or code in the memory 3220. The memory 3220 may include a storage medium such as Dynamic Random Access Memory (DRAM), read only memory (ROM), buffers, registers, cache, flash memory, hard disk drives, solid-state drives, or the like. The memory 3220 may store data 3222 such as frames, frame structures, frame headers, etc., and may also include code to generate, scramble, encode, decode, parse, and interpret MAC frames and/or PHY frames and PPDUs.

The processor(s) 3210 may couple with a clock 3212 to track time and to track an offset for a time associated with operations of the network 3290. For instance, when the patient device 3200 associates with the network 3290, an access point of the network 3290 may provide the patient device 3200 with the time of the network 3290 and the patient device 3200 may store an offset representing the different between the clock 3212 and the time on the network 3290 at the time of transmission of a frame including the time of the network 3290.

The patient device 3200 may also include a wireless communications interface (I/F) 3230. The wireless communications I/F 3230 may include baseband logic circuitry 3240 and a wireless network interface 3252. The baseband processing circuitry 3240 may include a baseband processor and/or one or more circuits to implement a station management entity and the station management entity may interact with a MAC layer management entity to perform MAC layer functionality and a PHY management entity to perform PHY functionality. In such examples, the baseband processing circuitry 3048 may interact with processor(s) 3210 to coordinate higher layer functionality with MAC layer and PHY functionality.

In the present example, the baseband processing circuitry 3240 includes Bluetooth logic circuitry 3242 and wireless local area network (WLAN) logic circuitry 3244. The WLAN logic circuitry 3244 may include a memory, such as, for example, a set of RAM arrays in a Fast Fourier Transform or Inverse Fast Fourier Transform block (not shown) of the WLAN baseband processing circuitry 3244. Each of the BT logic circuitry 3242 and WLAN logic circuitry 3244 may further include one or more processors and control logic to process the signals received from the corresponding WLAN or BT receive signal path of the BT/WLAN radio 3254, and to also generate corresponding WLAN or BT baseband signals for the transmit signal path of the BT/WLAN radio 3254. Each of the BT logic circuitry 3242 and WLAN logic circuitry 3244 may further include physical layer (PHY) and MAC layer circuitry and may further interface with a device for generation and processing of the baseband signals and for controlling operations of the BT/WLAN radio 3254.

BT/WLAN radio 3254 as shown may include WLAN radio IC circuitry and BT radio IC circuitry. The WLAN radio integrated circuit (IC) circuitry may include a receive signal path which may include circuitry to down-convert WLAN radio frequency (RF) signals received from the BT/WLAN front end module (FEM) 3256 and provide baseband signals to WLAN logic circuitry 3244. BT radio IC circuitry may in turn include a receive signal path which may include circuitry to down-convert BT RF signals received from the BT/WLAN FEM 3256 and provide baseband signals to BT logic circuitry 3242. WLAN radio IC circuitry may also include a transmit signal path which may include circuitry to up-convert WLAN baseband signals provided by the WLAN logic circuitry 3244 and provide WLAN RF output signals to the BT/WLAN FEM 3256 for subsequent wireless transmission by the one or more antennas 3258. BT radio IC circuitry may also include a transmit signal path which may include circuitry to up-convert BT baseband signals provided by the BT logic circuitry 3242 and provide BT RF output signals to the BT/WLAN FEM 3256 for subsequent wireless transmission by the one or more antennas 3258. Although the BT/WLAN radio 3254 is shown as the same circuitry, examples are not so limited, and include within their scope the use of more than one radio IC circuitries (not shown) that include distinct transmit signal paths and/or distinct receive signal paths for WLAN and BT signals, or the use of one or more radio IC circuitries where at least some of the radio IC circuitries transmit and/or receive signal paths for WLAN signals, BT signals, or a combination thereof.

The wireless communications I/F 3230 may facilitate communications by stations (STAs) such as the patient device 120, the server 130, and HCP device 140 in accordance with versions of Institute of Electrical and Electronics Engineers (IEEE) 802.11 standards for wireless communications such as IEEE P802.11be™/D1.0, May 2021; IEEE 802.11-2020, December 2020; IEEE P802.11ax™- 2021, IEEE P802.11ay™-2021, IEEE P802.11az™/D3.0, IEEE P802.11ba™-2021, IEEE P802.11bb™/D0.4, IEEE P802.11bc™/D1.02, and IEEE P802.11bd™/D1.1.

In some examples, the baseband processing circuitry 3240 may interact with one or more analog devices to perform PHY functionality such as descrambling, decoding, demodulating, and the like. In other examples, the baseband processing circuitry 3240 may execute code to perform one or more of the PHY functionality such as descrambling, decoding, demodulating, and the like.

The patient device 3000 may receive the one or more MAC frames with the prescription and instructions for micromotions at the antennas 3258, which pass the signals along to the BT/WLAN FEM 3256. The BT/WLAN FEM 3256 may amplify and filter the signals and pass the signals to the BT/WLAN radio 3254. The BT/WLAN radio 3254 may filter the carrier signals from the signals and determine if the signals represent a PPDU. If so, analog circuitry of the wireless network I/F 3252 or physical layer functionality implemented in the baseband processing circuitry 3240 may demodulate, decode, descramble, etc. the PPDU. The baseband processing circuitry 3240 may identify, parse, and interpret the one or more MAC frames from one or more PPDUs.

The processor(s) 3210 may execute an app to perform the prescription and instructions for micromotions received by the patient device 3200 via communication with one or more motor controller(s) 3260 of an automatic bone alignment device such as the automatic, external bone alignment device 115 shown in FIG. 1A. In some examples, the one or more motor controller(s) 3260 may include or be coupled with wireless communications circuitry to send and receive BT communications. In some examples, the one or more motor controller(s) 3260 may include or be coupled with wireless communications circuitry to send and receive WLAN communications. In some examples, the one or more motor controller(s) 3260 may include or be coupled with wireless communications circuitry to send and receive WLAN and/or BT communications.

In some examples, the patient device 320 may send commands to the motor controller(s) 3260 to perform the micromotions in conjunction with adjustments from an adjustment schedule of the prescription. In some examples, the commands detail movements for direct current (DC) motors such as steps for a step motor to cause the DC motors attached to the motor controllers to perform the micromotions. In other examples, the patient device 3200 may send commands that generally describe the micromotions and one or more on-board processing circuitries may execute the micromotions via logic or code stored in memory locally with the motor controller(s) 3260.

In some examples the circuitry described in FIG. 3C may be found within or connected to the external fixator mounted to the patient. As such the external fixator may receive data including prescription and micromotion instructions from the network 3290, follow the instructions, and send data concerning progress, system status, and the like back to the network 3290. Instructions from the network 3290 may require the external fixator be continuously connected to the network or may allow the external fixator to operate according to the instructions even when a network connection is lost.

FIG. 3D illustrates an example of an automatic bone alignment device 3305 comprising a wireless communications interface coupled with one or more automatic struts and integrated with removable geared-motor assemblies 3310 to communicate with correction logic circuitry of the patient device 3200 shown in FIG. 3A. In use, the removable geared-motor assemblies 3310 are arranged and configured to engage, attach, couple, etc. to the manually adjustable struts 3320 of the automatic bone alignment device 3305. Thus arranged, the automatic bone alignment device 3305 can be operated in and switched between two modes or configurations of operation. In the first mode or configuration of operation, the struts 3320 may be manually adjustable. In the second mode or configuration of operation, a geared-motor assembly 3310 may be attached to one or more of the manually adjustable struts 3320 to enable motorized and/or automated adjustment of the struts 3320 and rings 3330 and 3240.

In use, the geared-motor assemblies 3310 are coupled to the manually adjustable struts 3320 of the automatic bone alignment device 3305. In one example, the geared-motor assemblies 3210 may be coupled to the manually adjustable struts 3220 after surgery in clinic by, for example, a primary care provider. Alternatively, the geared-motor assemblies 3310 may be coupled to the manually adjustable struts 3320 at any time and by anyone. Once coupled, the geared-motor assemblies 3310 may facilitate motorized and/or automated adjustments such as, for example, semi-continuous actuation. For example, in one example, the geared-motor assemblies 3310 may enable motorized adjustments to be made autonomously once provided with instructions. Thus arranged, the automatic bone alignment device 3305 and/or system architecture may be arranged and configured to automatically adjust the motorized struts according to the prescription and micromotions of a treatment plan (e.g., automatically adjust the plurality of struts without patient intervention). Alternatively, and/or in addition, the automatic bone alignment device 3305 and/or system architecture may be arranged and configured to require patient and/or caregiver activation via a companion app on the patient device 3200 such as a smartphone, a tablet, or other computing system to begin the process of automatically adjusting the struts according to the treatment plan. For example, the automatic bone alignment device 3305 may be arranged to intermittently auto-adjust the motorized struts at predetermined times according to the adjustment schedule of the prescription for the treatment plan as well as perform micromotions during or between the automatic adjustments. Alternatively, the automatic bone alignment device 3305 may be arranged to intermittently auto-adjust the motorized struts at selected times of a schedule included in the instructions for the micromotions when convenient and/or when selected for or by the patient.

FIG. 3E illustrates an example of an automatic strut 3320 comprising the geared-motor assembly 3310 coupled with the strut 3320. In some examples, the geared-motor assemblies 3310 may each include an enclosure or housing 3430, a coupling mechanism 3440 for coupling the geared-motor assembly 3310 to the strut 3430, a motor 3420, a torque transferring mechanism 3410 (e.g., a transmission or gears for transferring rotation from the motor 3420 to the strut 3320), and all necessary components and circuity so that activation of the motor 3420 moves the strut 3320. For example, as shown in FIG. 3F, the gear-motor assemblies 3310 may include a control circuit 3500 with one or more microprocessors 3510, one or more sensors 3540 such as, for example, positional sensors to monitor the length of the struts, force sensors such as load sensors or an accelerometer for providing biomechanical feedback during bone healing, acoustic emission or vibration sensor for fault level detection in the gear train, a power usage sensor such as a current sensor to monitor instantaneous and/or average power consumption, and/or the like. The gear-motor assemblies 3310 may also include a communication chip 3550 with an internal or external antenna for facilitating communication and/or transfer of data, a power supply 3520 such as, for example, a battery, a charging circuit 3530, etc.

Thus arranged, in accordance with one or more features of the present disclosure, a number of advantages are achieved. For example, by utilizing detachable geared-motor assemblies 3310, motorized and/or automated adjustments of an automatic bone alignment device can be achieved. In use, the detachable geared-motor assemblies 3310 are arranged and configured to engage a manually adjustable strut 3320 in an outpatient setting thus enabling the automatic bone alignment device to be operated in two different modes or configurations: (a) a standard, manual adjustment mode where the lengths of the struts 3320 can be adjusted by manual rotation of a threaded adjustment nut and (b) motorized and/or automated adjustment via the detachable geared-motor assemblies 3310.

In addition, in accordance with some examples, by arranging the geared-motor assemblies 3310 as self-contained units or devices incorporating wireless, self-powered, and incorporating their own microprocessors, the geared-motor assemblies 3310 are arranged and configured as a self-contained unit including all of the necessary components and circuity to control each strut according to the prescribed treatment plan, the geared-motor assemblies eliminate the need for any external cables or wires that could snag during use and eliminate the need for incorporating a centralized master control unit onto one of the platforms of the automatic bone alignment device thereby reducing bulk and safety risk to the patient (e.g., self-containment of the control circuitry, wireless communication chip, and power source within geared-motor assemblies negate the need for cables and a centralized master control unit positioned elsewhere on the spatial frame along with any needed cables or wires).

In addition, by utilizing detachable geared-motor assemblies, existing features of the manually adjustable struts are retained. That is, with the geared-motor assemblies detached from the manually adjustable struts, operation of the struts is unaffected. For example, if the manually adjustable strut incorporates a quick adjustment feature (e.g., a quick adjustment nut) to enable manual lengthening of the strut without rotating the threaded nut or rod, such adjustment feature is retained thus enabling faster adjustment during, for example, initial setup in the operating room. Moreover, the detachable geared-motor assemblies provide an offset motor design allowing greater application or use. For example, by incorporating an offset motor design, a shorter minimum strut length can be achieved (approximately 80 mm), which allows the struts to be used for correcting deformities in, for example, children with shorter limbs.

As previously mentioned, in one example, when arranged in a spatial frame, the geared-motor assemblies may be arranged and configured to wirelessly exchange data, instructions, etc. with an external computing system such as, for example, a smartphone, a tablet, a computer, etc. running a companion APP. However, it is envisioned that the geared-motor assemblies may exchange data with an external computing system by any now known or hereafter developed system. For example, in one example, each of the geared-motor assemblies may include a communication interface to exchange data over a wired connection.

With reference to FIG. 3F, the geared-motor assembly 3310 includes a control circuit 3500 (e.g., a printed-circuit board (PCB)), a microcontroller 3510, a wireless communication chip 3550, a power supply 3520 such as, for example, one or more batteries, and a charging circuit 3530. The electronics and the power source being housed with the motor 3420 inside the housing 3430. In use, when properly coupled to each of the struts 3320, the geared-motor assemblies 3310 facilitate motorized and/or automated adjustment of the strut 3320. In addition, the geared-motor assemblies 3310 may be coupled (e.g., wirelessly coupled) to an external computing system running, for example, a companion APP.

In some examples, the geared-motor assemblies 3310 may include an IP-68 rated housing manufactured from any suitable material including, for example, a metal or metal alloy, a polymer, a light-weight material such as PEEK, nylon, aluminum, etc. In addition, the housing may be manufactured via any now known or hereafter developed technique such as, for example, injection molding, additive manufacturing, etc.

In use, the geared-motor assembly 3310 can be mounted to the manual struts 3320 via a coupling mechanism 3440, which can be arranged in any suitable mechanism now known or hereafter developed to couple or mount the geared-motor assemblies 3310 to the struts 3320 including, for example, clips, sleeves, magnets, straps, etc. Preferably, in some examples, the coupling mechanism 3440 enables easy attachment and detachment of the geared-motor assembly 3310 from the strut 3320 to facilitate a change in mode between manual and automated adjustment.

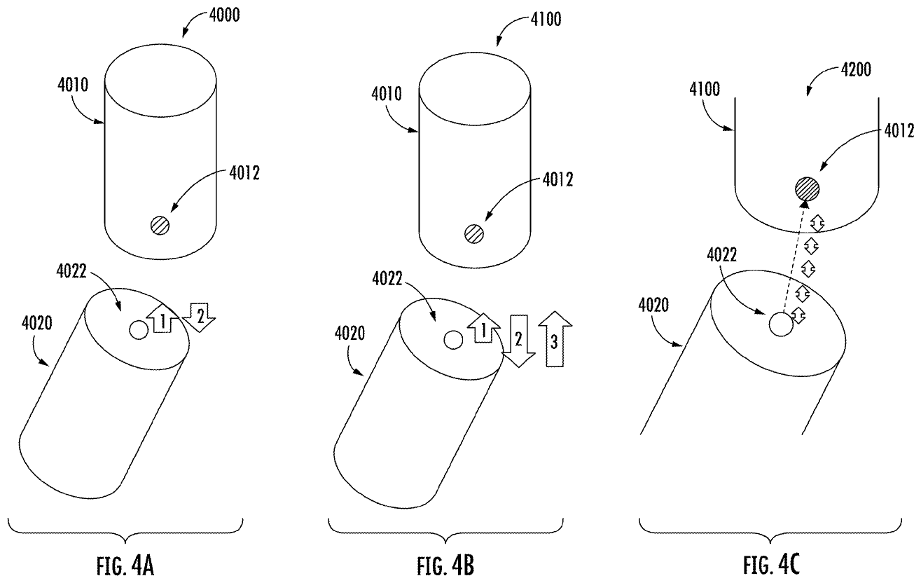

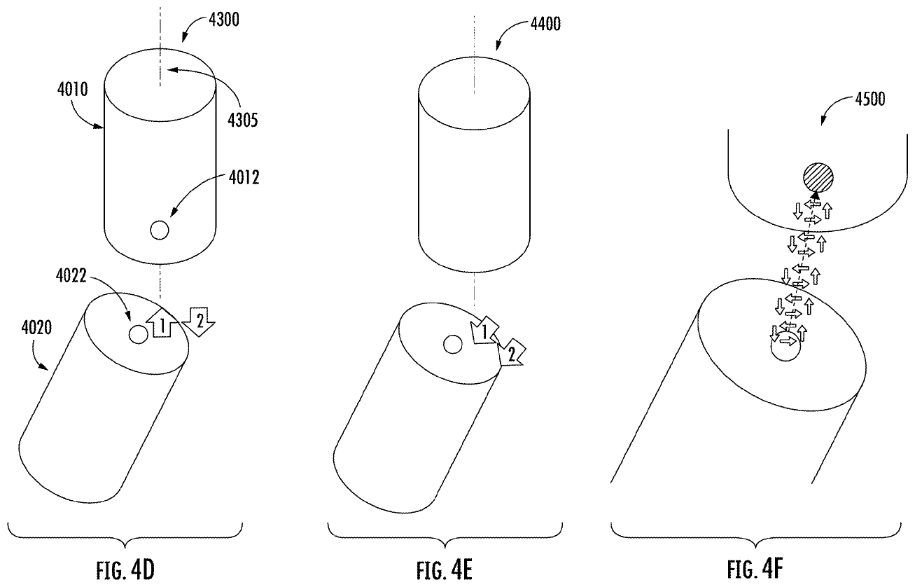

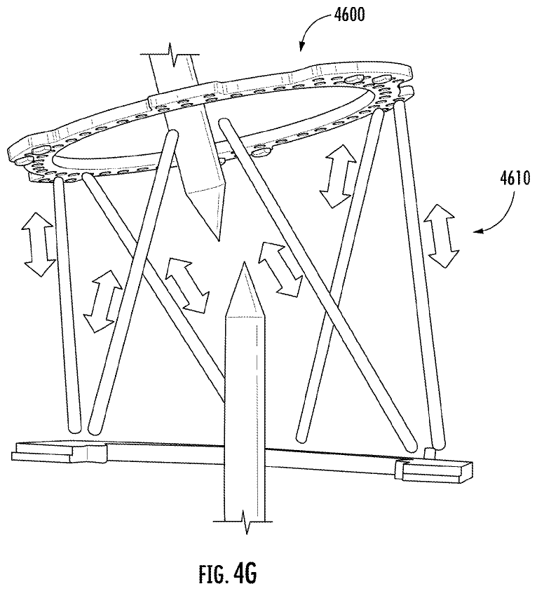

FIG. 4A-G illustrate examples of patterns of sets of micromotions. FIG. 4A illustrates an example of forward direction (1) and reverse direction (2) micromotions of a moving bone segment 4020. A static bone segment may have a point 4012 that is a destination location according to a treatment plan for a point 4022 on the moving bone segment 4020. In some examples, the automated external fixation device component(s) may follow instructions for pulsed micromotions such that the moving bone segment moves a set distance away from a starting point and then returns. The starting point may change after each programmed adjustment according to the prescription of adjustments.

FIG. 4B illustrates an example of the micromotions programmed to pulse the automated external fixation device component(s) such that the moving bone segment moves a set distance from a starting point in one direction (1) and then a set distance in the opposite direction (2). The set distances may or may not be equal. However, in most cases the micromotion will oscillate around a static starting point 4022 on the moving bone segment 4020 until the starting point 4022 moves (3) according to the prescriptions of adjustments.

FIG. 4C illustrates an example of the micromotions programmed to pulse the automated external fixation device component(s) while following a prescription of adjustments such that the moving bone segment bone segment/hardware do not stray farther than intended from the original prescription path, or, in other words, remains within the correction path boundaries even with the introduction of planned micromotions.

FIG. 4D illustrates an example of the micromotions programmed to pulse the automated external fixation device component(s). The rate of micromotions may affect bone quality. Rate is controlled in two ways in the various examples. Firstly, the rate of the micromotions is controllable. Users may set the frequency of the desired micromotions. For example, the automated external bone alignment device components may be programmed to move the moving bone segment 4020 a distance away (1) from the starting point and back (2) to the starting point at, e.g., 1 Hz. Secondly, the rest interval may be programmed into the micromotion schedule to accomplish intermittent loading schemes. For example, the automated external bone alignment device components may be programmed to move the moving bone segment 4020 a distance away (1) from the starting point and back (2) to the starting point at, e.g., 1 Hz for, e.g., 30 minutes, followed by, e.g., 30 minutes of rest.

Furthermore, the direction of micromotion is very important. The correction logic circuitry for an external bone alignment device may assume or require users to input the direction of the mechanical axis of the static (reference) bone segment 4010. This allows the correction logic circuitry to develop a 3D coordinate system. The directions of programmed micromotion may be specified relative to such an axis. In many situations, it is ideal to introduce micromotions of the moving bone segment parallel to the mechanical axis of the static bone segment 4010 within the bounds of the correction path defined by the prescription of adjustments.

FIG. 4E illustrates an example 4400 of the micromotions programmed to pulse the automated external fixation device component(s). In some examples, such as the example 4400, the correction logic circuitry may introduce micromotions of the moving bone segment 4010 along a path (1) and (2) that is not parallel to the mechanical axis 4305 of the static bone segment 4010. In the present example, the micromotion is introduced along the path (1) and (2) along the mechanical axis of the moving bone segment 4020. Furthermore, in some examples, the correction logic circuitry may introduce the micromotion at a defined trajectory that is not parallel to either the moving bone segment 4020 or the static bone segment 4020.

FIG. 4F illustrates an example 4500 of the micromotions programmed to pulse the automated external fixation device component(s) such that the micromotions arc programmed in any form of orbital path within the boundaries of the correction path.

FIG. 4G illustrates an example 4600 of the micromotions programmed to pulse the automated external bone alignment device component(s) such that the micromotions occur along the axis of the external bone alignment components such as along the axis of struts. With some external bone alignment devices, this may be completely independent of the axes of the bone segments. In the case of a hexapod bone alignment device, each strut may be actuated 4610 at the same or at different rates and/or magnitudes.

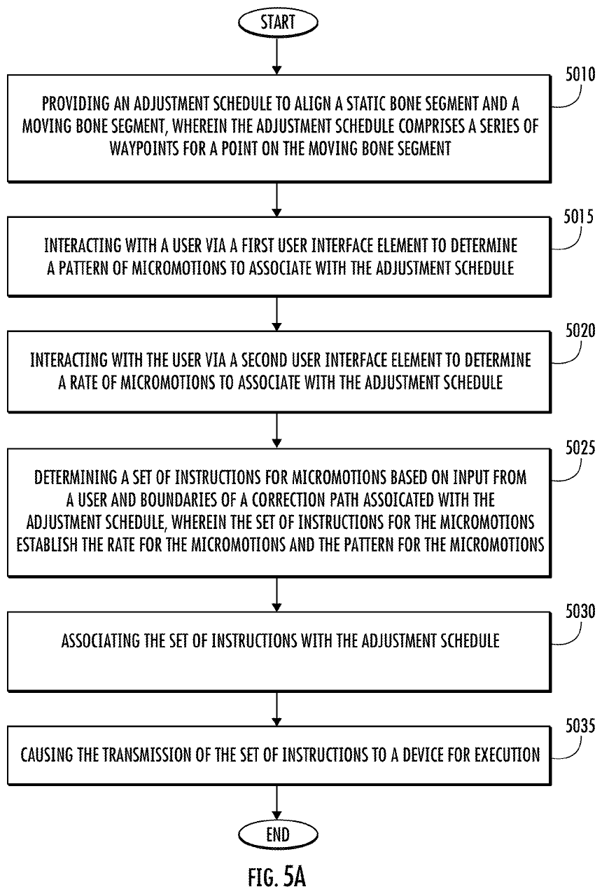

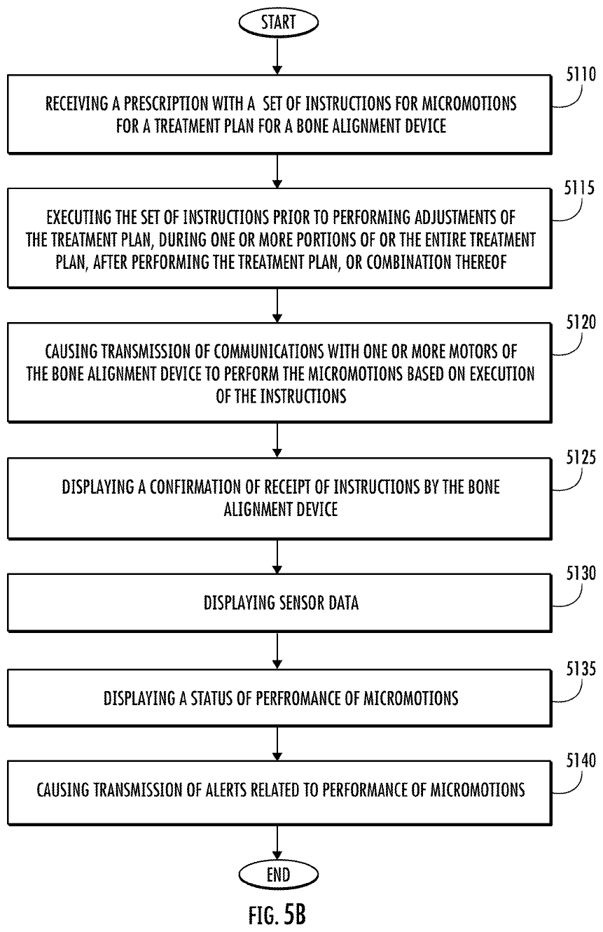

FIG. 5A depicts a flowchart of an example of a process 5000 to dynamize a bone alignment device. The process 5000 starts with providing an adjustment schedule to align a static bone segment and a moving bone segment, wherein the adjustment schedule includes a series of waypoints for a point on the moving bone segment (element 5010). For instance, a server such as the server 130 in FIG. 1A may include correction logic circuitry to generate an adjustment schedule of the prescription based on input information from a user. In some examples, the correction logic circuitry may receive the adjustment schedule and the user may interact with the correction logic circuitry to input information to define micromotions for implementation in conjunction with the prescription. In some examples, the correction logic circuitry may interact with the user to obtain input information to define micromotions for implementation in conjunction with the adjustment schedule while the user enters information to create the adjustment schedule. In some examples, the user may enter information such as user preference settings to define micromotions for implementation in conjunction with the adjustment schedule before the user enters information to create the adjustment schedule.

The process 5000 may involve interacting with a user via a first user interface element to determine a pattern of micromotions to associate with the adjustment schedule (element 5015). In many examples, the process 5000 may present a GUI with user interface elements to receive information about the micromotions from the user such as the pattern for the micromotions. In many examples, the micromotions do not have to only pulse in opposite directions. In such examples, the micromotions may move the moving bone segment in any direction or even in any pattern such as conical patterns, circular patterns, spiral patterns, uneven or non-symmetrical patterns, torsional patterns, and/or the like. Dynamization may be applied unevenly to stimulate bone growth on one side of the patient anatomy. For instance, dynamization may be applied to 2 or 4 struts of 6 struts to provide more micromotion on one side of the anatomy where healing may be lagging behind the other side of the anatomy. As another example, the process may implement torsional dynamization, where the micromotions may rotate one ring relative to the other of a hexapod bone alignment device. Such torsional micromotion may target anatomy and may lead to improved healing.

The process 5000 may involve interacting with the user via a second user interface element to determine a rate of the micromotions to associate with the adjustment schedule (element 5020). In some examples, the process may involve provision of a data entry field that includes a default value for the rate. In such examples, the user may confirm selection of the default value, edit the default value, enter a new value to replace the default value, select a different user preference for the rate, or select a rate from a list of rates.

After gathering information from the user and/or user preferences, the process 5000 may involve determining a set of instructions for micromotions based on input from a user and boundaries of a correction path associated with the adjustment schedule, wherein the set of instructions for the micromotions establish the rate for the micromotions and the pattern for the micromotions (element 5020). The process may involve identifying the instructions from one or more sets of instructions maintained in a library of instructions or instructions in memory that correlate with the micromotions identified by the user. In some examples, the process 5000 may involve generating compiled instructions for execution.

After determining the set of instructions for the micromotions, the process 5000 may associate the set of instructions with the adjustment schedule of a prescription (element 5030) and cause transmission of the set of instructions to the automated bone alignment device for execution (element 5035). For instance, the process 5000 may pass one or more sets of instructions for micromotions determined by the user to a communications interface to transmit the prescription along with the instructions for the micromotions to an intermediary device such as a mobile phone, tablet, or other computing system and then the automated bone alignment device or to the automated bone alignment device directly.

FIG. 5B depicts a flowchart of an example of a process 5100 to dynamize a bone alignment device. The process 5100 starts with receiving a prescription with a set of instructions for micromotions for a treatment plan for a bone alignment device (element 5110). For instance, a server such as the server 130 in FIG. 1A may include correction logic circuitry to generate a set of instructions for micromotions to implement in conjunction with a prescription based on input information from a user.

The process 5100 may involve executing the set of instructions prior to performing adjustments of the treatment plan, during one or more portions of or the entire treatment plan, after performing the treatment plan, or combination thereof (element 5115). A prescription may include a set of adjustments to be performed in accordance with a schedule such as one adjustment per day and the instructions, when executed, may cause the patient's automatic bone alignment device to implement the micromotions at times before, during, and/or after performance of one or all the adjustments in accordance with the rest period and/or schedule information provided by the user for the micromotions.

In some examples, the process 5100 may cause transmission of communications between an intermediary patient device such as a mobile phone, tablet, or other computing system with one or more motors of the bone alignment device to perform the micromotions based on execution of the instructions (element 5120). When executing the instructions for the set of micromotions, the instructions may cause a patient device, to send commands to an automatic bone alignment device to perform the micromotions. In some examples, the patient device may send commands to individual adjustment components of the bone alignment device (such as struts). In some examples, the patient device may send commands to a centralized controller for the adjustment components of the bone alignment device.

In some examples, the process 5100 may display a confirmation of receipt of instructions by the bone alignment device (element 5125). After sending one or more commands to the bone alignment device to perform adjustments and/or micromotions, the bone alignment device may respond with confirmations that each of the one or more commands were received.

In some examples, the bone alignment device may also provide sensor data to the patient device in response to performance of the commands and the patient device may display the sensor data on a display to a user of the patient device (element 5130). The sensor data may include data to verify the proper operation of the adjustable elements such as sensor data related to the power source, forces applied, strain applied, torque applied, movement of the adjustable component, and/or the like. The sensor data may be transmitted again from the patient device 120 to a server 130 or HCP device 130 shown in FIG. 1A.

In addition to or as an alternative to displaying sensor data, the device or devices receiving data from the automated bone alignment device may display a status of the performance of the micromotions based on feedback from the bone alignment device (element 5135). For instance, the bone alignment device may provide a status of the micromotions for feedback and so that status may be displayed for the micromotions instantaneously, continuously, periodically, and/or during the performance of the micromotions.

In some examples, addition to or as an alternative to displaying sensor data and/or a status of the micromotions, the patient device may cause transmission of alerts related to the performance of the micromotions (element 5140) such as alerts that describe inadequate performance and/or a failure of performance of one or more of the micromotions. For example, if sensor data received from one of the sensors exceeds a threshold for that sensor, the bone alignment device may stop performance of the micromotions and may transmit a corresponding alert to the patient device. Furthermore, the patient device may, for some alerts, transmit the alerts to the medical provider such as the surgeon by transmitting a corresponding alert to the HCP device 140 and/or the server 130 shown in FIG. 1A.

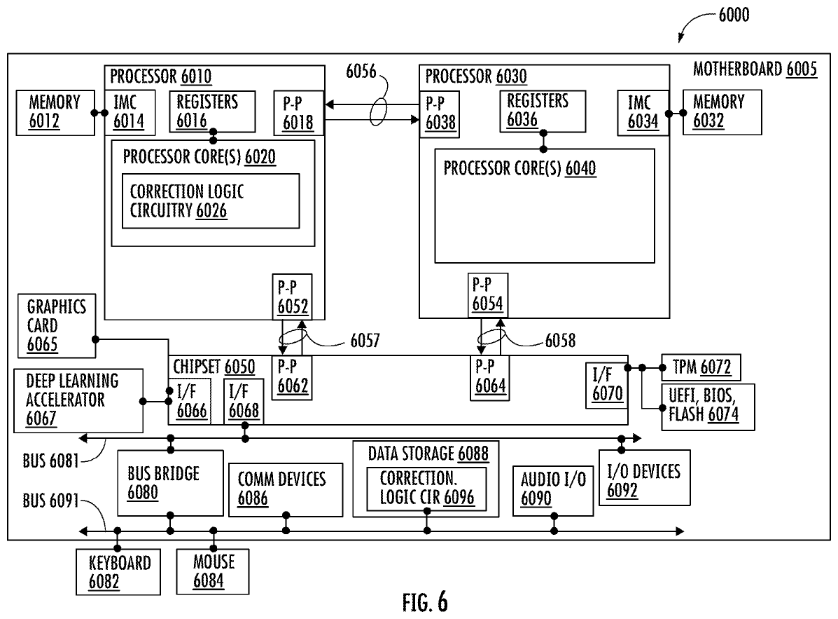

FIG. 6 illustrates an example of a system 6000. The system 6000 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar examples may include, e.g., entertainment devices such as a portable music player or a portable video player, a smart phone or other cellular phone, a telephone, a digital video camera, a digital still camera, an external storage device, or the like. Further examples implement larger scale server configurations. In other examples, the system 6000 may have a single processor with one core or more than one processor. Note that the term “processor” refers to a processor with a single core or a processor package with multiple processor cores.

As shown in FIG. 6, system 6000 includes a motherboard 6005 for mounting platform components. The motherboard 6005 is a point-to-point interconnect platform that includes a first processor 6010 and a second processor 6030 coupled via a point-to-point interconnect 6056 such as an Ultra Path Interconnect (UPI). In other examples, the system 6000 may be of another bus architecture, such as a multi-drop bus. Furthermore, each of processors 6010 and 6030 may be processor packages with multiple processor cores including processor core(s) 6020 and 6040, respectively. While the system 6000 is an example of a two-socket (2S) platform, other examples may include more than two sockets or one socket. For example, some examples may include a four-socket (4S) platform or an eight-socket (8S) platform. Each socket is a mount for a processor and may have a socket identifier. Note that the term platform refers to the motherboard with certain components mounted such as the processors 6010 and the chipset 6050. Some platforms may include additional components and some platforms may only include sockets to mount the processors and/or the chipset.

The first processor 6010 includes an integrated memory controller (IMC) 6014 and point-to-point (P-P) interconnects 6018 and 6052. Similarly, the second processor 6030 includes an IMC 6034 and P-P interconnects 6038 and 6054. The IMC's 6014 and 6034 couple the processors 6010 and 6030, respectively, to respective memories, a memory 6012 and a memory 6032. The memories 6012 and 6032 may be portions of the main memory (e.g., a dynamic random-access memory (DRAM)) for the platform such as double data rate type 3 (DDR3) or type 4 (DDR4) synchronous DRAM (SDRAM). In the present example, the memories 6012 and 6032 locally attach to the respective processors 6010 and 6030. In other examples, the main memory may couple with the processors via a bus and shared memory hub.

The processors 6010 and 6030 include caches coupled with each of the processor core(s) 6020 and 6040, respectively. In the present example, the processor core(s) 6020 of the processor 6010 include a correction logic circuitry 6026 such as the correction logic circuitry discussed in conjunction with FIGS. 1-5. The correction logic circuitry 6026 may represent circuitry configured to implement the functionality to adjust a correction path for bone alignment device or bone segments connected to a bone alignment device to generate a prescription of adjustments with micromotions for a bone alignment device to correct a bone deformity within the processor core(s) 6020 or may represent a combination of the circuitry within a processor and a medium to store all or part of the functionality of the comprehensive logic circuitry 6026 in memory such as cache, the memory 6012, buffers, registers, and/or the like. In several examples, the functionality of the correction logic circuitry 6026 resides in whole or in part as code in a memory such as the correction logic circuitry 6096 in the data storage unit 6088 attached to the processor 6010 via a chipset 6050 such as the correction logic circuitry discussed in FIGS. 1-5. The functionality of the correction logic circuitry 6026 may also reside in whole or in part in memory such as the memory 6012 and/or a cache of the processor. Furthermore, the functionality of the correction logic circuitry 6026 may also reside in whole or in part as circuitry within the processor 6010 and may perform operations, e.g., within registers or buffers such as the registers 6016 within the processor 6010, or within an instruction pipeline of the processor 6010.

In other examples, more than one of the processors 6010 and 6030 may include functionality of the correction logic circuitry 6026 such as the processor 6030 and/or the processor within the deep learning accelerator 6067 coupled with the chipset 6050 via an interface (I/F) 6066. The I/F 6066 may be, for example, a Peripheral Component Interconnect-enhanced (PCI-e).

The first processor 6010 couples to a chipset 6050 via P-P interconnects 6052 and 6062 and the second processor 6030 couples to a chipset 6050 via P-P interconnects 6054 and 6064. Direct Media Interfaces (DMIs) 6057 and 6058 may couple the P-P interconnects 6052 and 6062 and the P-P interconnects 6054 and 6064, respectively. The DMI may be a high-speed interconnect that facilitates, e.g., eight Giga Transfers per second (GT/s) such as DMI 3.0. In other examples, the processors 6010 and 6030 may interconnect via a bus.

The chipset 6050 may include a controller hub such as a platform controller hub (PCH). The chipset 6050 may include a system clock to perform clocking functions and include interfaces for an input/output (I/O) bus such as a universal serial bus (USB), peripheral component interconnects (PCIs), serial peripheral interconnects (SPIs), integrated interconnects (I2Cs), and the like, to facilitate connection of peripheral devices on the platform. In other examples, the chipset 6050 may include more than one controller hub such as a chipset with a memory controller hub, a graphics controller hub, and an I/O controller hub.

In the present example, the chipset 6050 couples with a trusted platform module (TPM) 6072 and the unified extensible firmware interface (UEFI), BIOS, Flash component 6074 via an interface (I/F) 6070. The TPM 6072 is a dedicated microcontroller designed to secure hardware by integrating cryptographic keys into devices. The UEFI, BIOS, Flash component 6074 may provide pre-boot code.

Furthermore, chipset 6050 includes an I/F 6066 to couple chipset 6050 with a high-performance graphics engine, graphics card 6065. In other examples, the system 6000 may include a flexible display interface (FDI) between the processors 6010 and 6030 and the chipset 6050. The FDI interconnects a graphics processor core in a processor with the chipset 6050.

Various I/O devices 6092 couple to the bus 6081, along with a bus bridge 6080 which couples the bus 6081 to a second bus 6091 and an I/F 6068 that connects the bus 6081 with the chipset 6050. In some examples, the second bus 6091 may be a low pin count (LPC) bus. Various devices may couple to the second bus 6091 including, for example, a keyboard 6082, a mouse 6084, communication devices 6086 and a data storage unit 6088 that may store code such as the correction logic circuitry 6096. Furthermore, an audio I/O 6090 may couple to second bus 6091. Many of the I/O devices 6092, communication devices 6086, and the data storage unit 6088 may reside on the motherboard 6005 while the keyboard 6082 and the mouse 6084 may be add-on peripherals. In other examples, some or all the I/O devices 6092, communication devices 6086, and the data storage unit 6088 are add-on peripherals and do not reside on the motherboard 6005.

FIG. 7 illustrates an example of a storage medium 7000 to dynamize a bone alignment device. Storage medium 7000 may include an article of manufacture. In some examples, storage medium 7000 may include any non-transitory computer readable medium or machine-readable medium, such as an optical, magnetic or semiconductor storage. Storage medium 7000 may store various types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

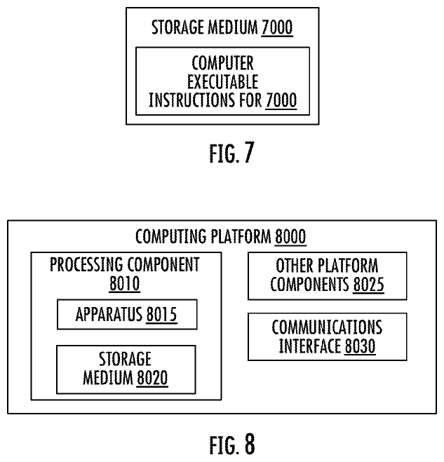

FIG. 8 illustrates an example computing platform 8000. In some examples, as shown in FIG. 8, computing platform 8000 may include a processing component 8010, other platform components or a communications interface 8030. According to some examples, computing platform 8000 may be implemented in a computing device such as a server in a system such as a data center or server farm that supports dynamizing a bone alignment device. Furthermore, the communications interface 8030 may include a wake-up radio (WUR) and may can wake up a main radio of the computing platform 8000.

According to some examples, processing component 8010 may execute processing operations or logic for apparatus 8015 described herein such as the correction logic circuitry discussed in conjunction with FIGS. 1-7. Processing component 8010 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 8020, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 8025 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 8030 may include logic and/or features to support a communication interface. For these examples, communications interface 8030 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2018, IEEE Standard for Ethernet, Published in August 2018 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.5, published in August 2021 (“the Infiniband Architecture specification”).

Computing platform 8000 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 8000 described herein, may be included or omitted in various examples of computing platform 8000, as suitably desired.

The components and features of computing platform 8000 may be implemented using any combination of discrete circuitry, ASICs, logic gates and/or single chip architectures. Further, the features of computing platform 8000 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 8000 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in examples.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

Various examples may be implemented using hardware elements, software elements, or a combination of both. In some examples, hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. In some examples, software elements may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

Some examples may include an article of manufacture or at least one computer-readable medium. A computer-readable medium may include a non-transitory storage medium to store logic. In some examples, the non-transitory storage medium may include one or more types of computer-readable storage media capable of storing electronic data, including volatile memory or non-volatile memory, removable or non-removable memory, erasable or non-erasable memory, writeable or re-writeable memory, and so forth. In some examples, the logic may include various software elements, such as software components, programs, applications, computer programs, application programs, system programs, machine programs, operating system software, middleware, firmware, software modules, routines, subroutines, functions, methods, procedures, software interfaces, API, instruction sets, computing code, computer code, code segments, computer code segments, words, values, symbols, or any combination thereof.

According to some examples, a computer-readable medium may include a non-transitory storage medium to store or maintain instructions that when executed by a machine, computing device or system, cause the machine, computing device or system to perform methods and/or operations in accordance with the described examples. The instructions may include any suitable type of code, such as source code, compiled code, interpreted code, executable code, static code, dynamic code, and the like. The instructions may be implemented according to a predefined computer language, manner, or syntax, for instructing a machine, computing device or system to perform a certain function. The instructions may be implemented using any suitable high-level, low-level, object-oriented, visual, compiled and/or interpreted programming language.

Some examples may be described using the expression “in one example” or “an example” along with their derivatives. These terms mean that a particular feature, structure, or characteristic described in connection with the example is included in at least one example. The appearances of the phrase “in one example” in various places in the specification are not necessarily all referring to the same example.

Some examples may be described using the expression “coupled” and “connected” along with their derivatives. These terms are not necessarily intended as synonyms for each other. For example, descriptions using the terms “connected” and/or “coupled” may indicate that two or more elements are in direct physical or electrical contact with each other. The term “coupled,” however, may also mean that two or more elements are not in direct contact with each other, but yet still co-operate or interact with each other.

In addition, in the foregoing Detailed Description, it can be seen that various features are grouped together in a single example for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code to reduce the number of times code must be retrieved from bulk storage during execution. The term “code” covers a broad range of software components and constructs, including applications, drivers, processes, routines, methods, modules, firmware, microcode, and subprograms. Thus, the term “code” may be used to refer to any collection of instructions which, when executed by a processing system, perform a desired operation or operations.

Logic circuitry, devices, and interfaces herein described may perform functions implemented in hardware and also implemented with code executed on one or more processors. Logic circuitry refers to the hardware or the hardware and code that implements one or more logical functions. Circuitry is hardware and may refer to one or more circuits. Each circuit may perform a particular function. A circuit of the circuitry may include discrete electrical components interconnected with one or more conductors, an integrated circuit, a chip package, a chip set, memory, or the like. Integrated circuits include circuits created on a substrate such as a silicon wafer and may include components. And integrated circuits, processor packages, chip packages, and chipsets may include one or more processors.

Processors may receive signals such as instructions and/or data at the input(s) and process the signals to generate the at least one output. While executing code, the code changes the physical states and characteristics of transistors that make up a processor pipeline. The physical states of the transistors translate into logical bits of ones and zeros stored in registers within the processor. The processor can transfer the physical states of the transistors into registers and transfer the physical states of the transistors to another storage medium.

A processor may include circuits to perform one or more sub-functions implemented to perform the overall function of the processor. One example of a processor is a state machine or an application-specific integrated circuit (ASIC) that includes at least one input and at least one output. A state machine may manipulate the at least one input to generate the at least one output by performing a predetermined series of serial and/or parallel manipulations or transformations on the at least one input.

While the present examples have described one or more features for use in an in-line motorized strut, it is envisioned that the one or more features may be used in a motorized strut having an offset motor design (e.g., longitudinal axis of the motor is offset from the longitudinal axis of the threaded rod). For example, by incorporating a secondary telescoping mechanism into a motorized strut having an offset motor design, the motorized strut could benefit from having a larger working length, meaning less strut changeouts and less inventory. As such, the present disclosure should not be limited to an in-line design unless specifically claimed.

Thus arranged, in accordance with the features of the present disclosure, the motorized struts serve to maximize the range (e.g., working range) of a motorized strut, and more preferably an in-line motorized strut. In use, the addition of an independent telescoping member allows quick, manual length adjustment in, for example, the operating room during initial setup, while not using any of the working length associated with rotation of the threaded rod. In addition, and/or alternatively, incorporation of a two-stage telescoping design allows for essentially twice the working length of a motorized strut. If combined, a motorized strut having a larger working length (e.g., 2× working range) and the capability to be manually lengthened in the operating room without using any of the working length can be provided.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

Further examples may include:

Example 1 is an apparatus to dynamize a bone alignment device, comprising memory and logic circuitry coupled with the memory to enable to the logic circuitry to provide an adjustment schedule to align a static bone segment and a moving bone segment, wherein the adjustment schedule comprises a series of waypoints for a point on the moving bone segment; receive via a first user interface element to determine a pattern of micromotions to associate with the adjustment schedule; receive via a second user interface element to determine a rate of the micromotions to associate with the adjustment schedule; determine a set of instructions for the micromotions, wherein the set of instructions for the micromotions establish the rate for the micromotions and the pattern for the micromotions; associate the set of instructions with the adjustment schedule; and cause transmission of the set of instructions to a device for execution during treatment in conjunction with the adjustment schedule, the execution of the set of instructions to cause the bone alignment device to automatically move the point of the moving bone segment in the micromotions within the boundaries of the correction path in accordance with the set of instructions. In Example 2, the apparatus of Example 1, further comprising a communication interface, wherein the logic circuitry comprises at least one processor coupled with the memory. In Example 3, the apparatus of Example 1, further comprising a third user interface element to determine a rest interval between application of the micromotions. In Example 4, the apparatus of Example 3, wherein the rest interval between application of the micromotions comprises a schedule, the schedule comprising a time of day, or wherein the rest interval between application of the micromotions comprises a period of time. In Example 5, the apparatus of Example 3, wherein the set of instructions for micromotions is determined based on input from a user, boundaries of a correction path associated with the adjustment schedule, or a combination thereof. In Example 6, the apparatus of Example 3, wherein periods of the micromotions and rest intervals occur with varying durations throughout a period of time. In Example 7, the apparatus of any one of Examples 1-6, the execution of the set of instructions to receive sensor data and cause the bone alignment device to adjust the micromotions in response, wherein adjustment of the micromotions comprises adjustment of the rate of the micromotions, a magnitude of the micromotions, a direction of the micromotions, or a combination thereof. In Example 8, the apparatus of Example 7, the execution of the set of instructions to receive the sensor data and cause the bone alignment device to stop the micromotions in response to the sensor data, wherein the sensor data reaches or exceeds a threshold for the sensor data. In Example 9, the apparatus of Example 7, wherein the sensor data comprises a force, a strain, a power usage, or a combination thereof. In Example 10 is a non-transitory computer-readable medium, comprising code to dynamize a bone alignment device, wherein the code when executed by a processor, causes the processor to perform operations to receive a prescription with a set of instructions for micromotions for a treatment plan for the bone alignment device; execute the set of instructions prior to performing adjustments of the treatment plan, during one or more portions of or all portions of the treatment plan, after performing the treatment plan, or combination thereof; and cause transmission of communications to one or more motor controller circuits of the bone alignment device to perform the micromotions based on execution of the instructions. In Example 11, the computer-readable medium of Example 10, the operations to further display a confirmation of receipt of instructions by the bone alignment device; display sensor data; display a status of performance of the micromotions; and cause transmission of alerts related to performance of the micromotions. In Example 12, the computer-readable medium of Example 10, the operations to further interact with a user via a third user interface element to determine a rest interval between application of the micromotions. In Example 13, the computer-readable medium of Example 12, wherein the rest interval between the application of the micromotions comprises a schedule, the schedule comprising a time of day. In Example 14, the computer-readable medium of Example 12, wherein the rest interval between the application of the micromotions comprises a period of time. In Example 15, the computer-readable medium of Example 12, wherein periods of the micromotions and rest intervals occur with varying durations throughout a period of time. In Example 16, the computer-readable medium of Example 10, the operations to further receive sensor data and adjust the micromotions in response, wherein adjustment of the micromotions comprises adjustment of the rate of the micromotions, a magnitude of the micromotions, a direction of the micromotions, or a combination thereof. In Example 17, the computer-readable medium of Example 16, the operations to further receive the sensor data and stop the micromotions in response to the sensor data, wherein the sensor data reaches or exceeds a threshold for the sensor data. In Example 18, the computer-readable medium of Example 16, wherein the sensor data comprises a force, a strain, a power usage, or a combination thereof. In Example 19, the computer-readable medium of Example 10, the operations to further interact with a user via a fourth user interface element to determine a portion of the adjustment schedule within which to integrate the set of instructions for the micromotions. In Example 20, the computer-readable medium of Example 19, wherein the portion of the adjustment schedule comprises a schedule. In Example 21, the computer-readable medium of Example 19, wherein the portion of the adjustment schedule comprises a pre-treatment portion, an initial treatment portion, a middle treatment portion, an end treatment portion, a post-treatment portion, or a combination thereof. In Example 22, the computer-readable medium of Example 10, wherein the pattern of the micromotions comprises a forward direction and a reverse direction, wherein the forward direction is a direction along a mechanical axis of a static bone segment connected to the bone alignment device from a starting point and the reverse direction is a direction opposite of the forward direction to move the point on a moving bone segment, connected to the bone alignment device, along the mechanical axis of the static bone segment to the starting point. In Example 23, the computer-readable medium of Example 10, wherein the pattern of the micromotions comprises a first direction and a second direction, wherein the first direction is away from a starting point and the second direction is a direction opposite of the first direction to move the point on a moving bone segment back to the starting point. In Example 24, the computer-readable medium of Example 10, wherein the pattern of the micromotions comprises a first direction and a second direction, wherein the first direction is away from a starting point and the second direction is a direction toward a waypoint of the adjustment schedule that is subsequent to the starting point. In Example 25, the computer-readable medium of Example 10, wherein the pattern of the micromotions comprises a first direction and a second direction, wherein the first direction is not parallel to a mechanical axis of a static bone segment. In Example 26, the computer-readable medium of Example 10, wherein the pattern of the micromotions comprises an orbital pattern, wherein the orbital pattern returns the point on a moving bone segment to the starting point. In Example 27, the computer-readable medium of Example 10, wherein the pattern of the micromotions comprises an orbital pattern, wherein the orbital pattern moves the point on a moving bone segment to a subsequent waypoint. In Example 28, the computer-readable medium of Example 10, wherein the pattern of the micromotions moves the point on a moving bone segment to a subsequent waypoint. In Example 29, the computer-readable medium of Example 10, wherein the pattern of the micromotions unevenly apply force and strain to a side of a static bone segment and a side of a moving bone segment to stimulate bone growth on one side of a patient anatomy. In Example 30, the computer-readable medium of Example 10, wherein the pattern of the micromotions apply a torsional micromotion to a static bone segment and a moving bone segment. In Example 31, the computer-readable medium of Example 10, wherein the pattern for application of the micromotions varies at different adjustments in the prescription. In Example 32, the computer-readable medium of Example 10, the operations to further interact with a user via a fourth user interface element to determine a schedule for application of the micromotions. In Example 33, the computer-readable medium of Example 10, the operations to further interact with a user via a fifth user interface element to determine a magnitude for application of the micromotions. In Example 34, the computer-readable medium of any one of Examples 10-23, the operations to further apply the micromotions to a portion of the adjustment schedule via an automated bone alignment device in response to the execution of the set of instructions, wherein the portion comprises a pre-treatment portion, an initial treatment portion, a middle treatment portion, an end treatment portion, a post-treatment portion, or a combination thereof.

Example 35 is a method for a graphical user interface to dynamize a bone alignment device, the method comprising providing an adjustment schedule to align a static bone segment and a moving bone segment, wherein the adjustment schedule comprises a series of waypoints for a point on the moving bone segment; interacting with a user via a first user interface element to determine a pattern of micromotions to associate with the adjustment schedule; interacting with the user via a second user interface element to determine a rate of the micromotions to associate with the adjustment schedule; determining a set of instructions for the micromotions based on input from the user and boundaries of a correction path associated with the adjustment schedule, wherein the set of instructions for the micromotions establish the rate for the micromotions and the pattern for the micromotions; associating the set of instructions with the adjustment schedule; and causing transmission of the set of instructions to a device for execution during treatment in conjunction with the adjustment schedule, the execution of the set of instructions to cause the bone alignment device to automatically move the point of the moving bone segment in the micromotions within the boundaries of the correction path in accordance with the set of instructions. In Example 36, the method of Example 35, further comprising interacting with the user via a third user interface element to determine a rest interval between application of the micromotions. In Example 37, the method of Example 36, wherein the rest interval between application of the micromotions comprises a schedule, the schedule comprising a time of day. In Example 38, the method of Example 36, wherein the rest interval between application of the micromotions comprises a period of time. In Example 39, the method of Example 36, wherein periods of the micromotions and rest intervals occur with varying durations throughout a period of time. In Example 40, the method of Example 35, further comprising receiving sensor data and adjusting the micromotions in response, wherein adjusting the micromotions comprises adjusting the rate of the micromotions, a magnitude of the micromotions, a direction of the micromotions, or a combination thereof. In Example 41, the method of Example 40, further comprising receiving the sensor data and stopping the micromotions in response to the sensor data, wherein the sensor data reaches or exceeds a threshold for the sensor data. In Example 42, the method of Example 40, wherein the sensor data comprises a force, a strain, a power usage, or a combination thereof. In Example 43, the method of Example 35, further comprising interacting with the user via a fourth user interface element to determine a portion of the adjustment schedule within which to integrate the set of instructions for the micromotions. In Example 44, the method of Example 43, wherein the portion of the adjustment schedule comprises a schedule. In Example 45, the method of Example 43, wherein the portion of the adjustment schedule comprises a pre-treatment portion, an initial treatment portion, a middle treatment portion, an end treatment portion, a post-treatment portion, or a combination thereof. In Example 46, the method of Example 35, wherein the pattern of the micromotions comprises a forward direction and a reverse direction, wherein the forward direction is a direction along a mechanical axis of the static bone segment from a starting point and the reverse direction Is a direction opposite of the forward direction to move the point on the moving bone segment along the mechanical axis of the static bone segment to the starting point. In Example 47, the method of Example 35, wherein the pattern of the micromotions comprises a first direction and a second direction, wherein the first direction is away from a starting point and the second direction is a direction opposite of the first direction to move the point on the moving bone segment back to the starting point. In Example 48, the method of Example 35, wherein the pattern of the micromotions comprises a first direction and a second direction, wherein the first direction is away from a starting point and the second direction is a direction toward a waypoint of the adjustment schedule that is subsequent to the starting point. In Example 49, the method of Example 35, wherein the pattern of the micromotions comprises a first direction and a second direction, wherein the first direction is not parallel to a mechanical axis of the static bone segment. In Example 50, the method of Example 35, wherein the pattern of the micromotions comprises an orbital pattern, wherein the orbital pattern returns the point on the moving bone segment to the starting point. In Example 51, the method of Example 35, wherein the pattern of the micromotions comprises an orbital pattern, wherein the orbital pattern moves the point on the moving bone segment to a subsequent waypoint. In Example 52, the method of Example 35, wherein the pattern of the micromotions moves the point on the moving bone segment to a subsequent waypoint. In Example 53, the method of Example 35, wherein the pattern of the micromotions unevenly apply force and strain to a side of the static bone segment and a side of the moving bone segment to stimulate bone growth on one side of a patient anatomy. In Example 54, the method of Example 35, wherein the pattern of the micromotions apply a torsional micromotion to the static bone segment and the moving bone segment. In Example 55, the method of Example 35, wherein the pattern for application of the micromotions varies at different adjustments in a prescription. In Example 56, the method of Example 35, further comprising interacting with the user via a fourth user interface element to determine a schedule for application of the micromotions. In Example 57, the method of Example 35, further comprising interacting with the user via a fifth user interface element to determine a magnitude for application of the micromotions. In Example 58, the method of Example 35, further comprising applying the micromotions to a portion of the adjustment schedule via an automated bone alignment device in response to the execution of the set of instructions, wherein the portion comprises a pre-treatment portion, an initial treatment portion, a middle treatment portion, an end treatment portion, a post-treatment portion, or a combination thereof. Example 57 is an apparatus including a means for performing any one of Examples 35-58, further comprising interacting with the user via a fifth user interface element to determine a magnitude for application of the micromotions.

1. An apparatus to dynamize a bone alignment device, comprising:
memory and
logic circuitry coupled with the memory to enable to the logic circuitry to:
provide an adjustment schedule to align a static bone segment and a moving bone segment, wherein the adjustment schedule comprises a series of waypoints for a point on the moving bone segment;
receive via a first user interface element to determine a pattern of micromotions to associate with the adjustment schedule;
receive via a second user interface element to determine a rate of the micromotions to associate with the adjustment schedule;
determine a set of instructions for the micromotions herein the set of instructions for the micromotions establish the rate for the micromotions and the pattern for the micromotions;
associate the set of instructions with the adjustment schedule; and
cause transmission of the set of instructions to a device for execution during treatment in conjunction with the adjustment schedule, the execution of the set of instructions to cause the bone alignment device to automatically move the point of the moving bone segment in the micromotions within the boundaries of the correction path in accordance with the set of instructions.

2. The apparatus of claim 1, further comprising a communication interface, wherein the logic circuitry comprises at least one processor coupled with the memory.

3. The apparatus of claim 1, further comprising a third user interface element to determine a rest interval between application of the micromotions.

4. The apparatus of claim 3, wherein the rest interval between application of the micromotions comprises a schedule, the schedule comprising a time of day or wherein the rest interval between application of the micromotions comprises a period of time.

5. The apparatus of claim 3, wherein the set of instructions for the micromotions is based on boundaries of a correction path associated with the adjustment schedule, input from a user, or a combination thereof.

6. The apparatus of claim 3, wherein periods of the micromotions and rest intervals occur with varying durations throughout a period of time.

7. The apparatus of claim 1, the execution of the set of instructions to receive sensor data and cause the bone alignment device to adjust the micromotions in response, wherein adjustment of the micromotions comprises adjustment of the rate of the micromotions, a magnitude of the micromotions, a direction of the micromotions, or a combination thereof.

8. The apparatus of claim 7, the execution of the set of instructions to receive the sensor data and cause the bone alignment device to stop the micromotions in response to the sensor data, wherein the sensor data reaches or exceeds a threshold for the sensor data.

9. The apparatus of claim 7, wherein the sensor data comprises a force, a strain, a power usage, or a combination thereof.

10. A non-transitory computer-readable medium, comprising code to dynamize a bone alignment device, wherein the code when executed by a processor, causes the processor to perform operations to:
receive a prescription with a set of instructions for micromotions for a treatment plan for the bone alignment device;
execute the set of instructions prior to performing adjustments of the treatment plan, during one or more portions of or all portions of the treatment plan, after performing the treatment plan, or combination thereof; and
cause transmission of communications to one or more motor controller circuits of the bone alignment device to perform the micromotions based on execution of the instructions.

11. The computer-readable medium of claim 10, the operations to further:
display a confirmation of receipt of instructions by the bone alignment device;
display sensor data;
display a status of performance of the micromotions; and
cause transmission of alerts related to performance of the micromotions.

12. The computer-readable medium of claim 10, the operations to further interact with a user via a third user interface element to determine a rest interval between application of the micromotions.

13. The computer-readable medium of claim 12, wherein the rest interval between the application of the micromotions comprises a schedule, the schedule comprising a time of day.

14. The computer-readable medium of claim 12, wherein the rest interval between the application of the micromotions comprises a period of time.

15. The computer-readable medium of claim 12, wherein periods of the micromotions and rest intervals occur with varying durations throughout a period of time.
